(19) United States
(12) Patent Application Publication
(10) Pub. No.: US 2017/0095257 A1
(43) Pub. Date: Apr. 6, 2017

(54) DEVICES AND METHODS FOR OCCLUDING AN ATRIAL APPENDAGE

(71) Applicant: AMSEL MEDICAL CORPORATION, Cambridge, MA (US)

(72) Inventors: Arnold Miller, Cambridge, MA (US); Raanan A. Miller, Cambridge, MA (US)

(21) Appl. No.: 15/380,245

(22) Filed: Dec. 15, 2016

Related U.S. Application Data
(63) Continuation-in-part of application No. 15/226,577, filed on Aug. 2, 2016.
Application No. 15/226,577 is a continuation-in-part of application No. 14/639,814, filed on Mar. 5, 2015.
Application No. 14/639,814 is a continuation-in-part of application No. 14/272,304, filed on May 7, 2014.
Application No. 14/272,304 is a continuation-in-part of application No. 13/857,424, filed on Apr. 5, 2013.
Application No. 13/857,424 is a continuation-in-part of application No. 13/348,416, filed on Jan. 11, 2012.
(60) Provisional application No. 62/203,655, filed on Aug. 11, 2015.
(60) Provisional application No. 62/298,724, filed on Feb. 23, 2016.
(60) Provisional application No. 62/361,547, filed on Jul. 13, 2016.
(60) Provisional application No. 62/306,932, filed on Mar. 11, 2016.
(60) Provisional application No. 62/303,071, filed on Mar. 3, 2016.
(60) Provisional application No. 62/299,662, filed on Feb. 25, 2016.
(60) Provisional application No. 62/299,444, filed on Feb. 24, 2016.
(60) Provisional application No. 62/276,042, filed on Jan. 7, 2016.
(60) Provisional application No. 62/267,883, filed on Dec. 15, 2015.
(60) Provisional application No. 62/267,883, filed on Dec. 15, 2015.
(60) Provisional application No. 62/084,989, filed on Nov. 26, 2014.
(60) Provisional application No. 61/948,241, filed on Mar. 5, 2014.
(60) Provisional application No. 61/820,589, filed on May 7, 2013.
(60) Provisional application No. 61/620,787, filed on Apr. 5, 2012.
(60) Provisional application No. 61/431,609, filed on Jan. 11, 2011.

(51) Int. Cl. A61B 17/12; A61B 17/122; A61B 17/00

(52) U.S. Cl. CPC A61B 17/12122; A61B 17/0057; A61B 17/12031; A61B 17/122; A61B 17/1222; A61B 2017/00632; A61B 2017/00623; A61B 2017/12054

(57) ABSTRACT

The present invention provides devices and methods for using occlusion devices to exclude an atrial appendage. The invention can be employed during open surgery or thoracoscopy. The invention generally involves placing a row of occlusion clamps at the edge of the left atrial appendage (LAA) to cut off flow from the rest of the heart. The LAA can be simply excluded with the occluders, or it can be subsequently resected.

### CROSS-REFERENCE TO RELATED APPLICATIONS

This application claims the benefit of and priority to U.S. Provisional Application Ser. No. 62/267,825, filed Dec. 15, 2015; U.S. Provisional Application Ser. No. 62/267,883, filed Dec. 15, 2015; U.S. Provisional Application Ser. No. 62/276,042, filed Jan. 7, 2016; U.S. Provisional Application Ser. No. 62/299,444, filed Feb. 24, 2016; U.S. Provisional Application Ser. No. 62/299,662, filed Feb. 25, 2016; U.S. Provisional Application Ser. No. 62/303,071, filed Mar. 3, 2016; U.S. Provisional Application Ser. No. 62/306,932, filed Mar. 11, 2016; and U.S. Provisional Application Ser. No. 62/361,547, filed Jul. 13, 2016; and

is a continuation-in-part of U.S. application Ser. No. 15/226,577, which claims the benefit of and priority to U.S. Provisional Application Ser. No. 62/203,655, filed Aug. 11, 2015, and U.S. Provisional Application Ser. No. 62/298,724, filed Feb. 23, 2016, and is a continuation-in-part of U.S. application Ser. No. 14/639,814, filed Mar. 5, 2015 (which claims the benefit of and priority to U.S. Provisional Application Ser. No. 62/084,989, filed Nov. 26, 2014), which is a continuation-in-part of U.S. application Ser. No. 14/272,304, filed May 7, 2014 (which claims the benefit of and priority to U.S. Provisional Application Ser. No. 61/948,241, filed Mar. 5, 2014, and U.S. Provisional Application Ser. No. 61/820,589, filed May 7, 2013), which is a continuation-in-part of U.S. application Ser. No. 13/857,424, filed Apr. 5, 2013 (which claims the benefit of and priority to U.S. Provisional Application Ser. No. 61/620,787, filed Apr. 5, 2012), which is a continuation-in-part of U.S. application Ser. No. 13/348,416, filed Jan. 11, 2012 (which claims the benefit of and priority to U.S. Provisional Application Ser. No. 61/431,609, filed Jan. 11, 2011);

the entire contents of each of which are incorporated by reference herein in their entirety.

### FIELD OF THE INVENTION

The invention generally relates to surgical methods for occlusion of hollow structures and tissue fixation, and more particularly to methods for occluding or resecting an atrial appendage.

### BACKGROUND

Embolic stroke is the one of the nation's leading mortality factors for adults, and is a major cause of disability. A common cause of embolic stroke is the release of thrombus formed in the left atrial appendage (“LAA”) resulting from atrial fibrillation. The LAA is a small windsock-like cavity that extends from the lateral wall of the left atrium generally between the mitral valve and the root of the left pulmonary vein. The LAA normally contracts with the left atrium during systole, thus preventing blood within the LAA from becoming stagnant. During atrial fibrillation, however, the LAA fails to vigorously contract due to the lack of synchronicity of the electrical signals in the left atrium. As a result, thrombus may form in the stagnant blood that pools within the LAA, which may subsequently be ejected into systemic circulation after a normal sinus rhythm is reinstituted.

Challenges exist for excluding or removing the LAA. For example, the LAA is extremely thin and friable, meaning that traditional methods of suturing or stapling often can result in bleeding. LAA procedures are typically performed when the patient is on a heart-bypass machine, and bleeding associated with the typical open or thorascopic methods of occlusion of the LAA generally becomes apparent once the patient is taken off the machine and the heart begins pumping again. If bleeding does occur through the suture or staple tissue incision points, the patient may need to be put back on the bypass machine to stop the bleeding which exposes the patient to significant additional risk and morbidity.

Known methods for preventing such problems with the LAA have drawbacks. For example, most of the previously-known percutaneous devices are designed for an ideal LAA anatomical structure, including a well-defined, symmetric, and typically circular ostium and expected depth and orientation of the LAA cavity. Other devices that employ a loop applied to the base of the LAA on the pericardial surface may abrade the pericardial surface, thus leading to potentially fatal cardiac pericarditis or pericardial tamponade. Other devices involve using expandable disks to clamp and collapse the LAA tissue, but these too rely on the LAA having reasonably symmetric and well-defined depth and anatomy and have issues around sealing at tissue puncture points.

### SUMMARY

The present disclosure involves devices and methods for using occlusion devices to exclude an atrial appendage and other related cardiac and vascular procedures. The devices and methods can be employed during open surgery or thoracoscopy. Occlusion devices described herein can be placed in a row at the edge of the left atrial appendage (LAA), for example, to cut off flow from the rest of the heart. Unlike prior art clips, the occluders can be precisely deployed at the desired locations, including in a non-linear pattern that follows the contours of tissue desired to be occluded and on varying tissue thicknesses where each locking element can accommodate a different thickness of tissue. The LAA can be simply excluded with the occluders, or it can be subsequently resected. The occlusion devices can also be used to close surgical incisions in fragile tissue such as the atria, which commonly experience post-operative bleeding with the use of regular sutures.

The invention also provides methods and devices for elevating or isolating the LAA from the rest of the heart in order to apply the occluders. Elevation and isolation can be performed using a laparoscopic device as described herein. It can be aided by using a sling made from synthetic material or animal tissue. The sling can be a noose-like or lasso-like structure of adjustable width that wraps around the base of the LAA and can be tightened with an adjustable tightening member to snugly fit around the LAA.

While procedures involving the LAA are described extensively throughout the present disclosure, it is to be understood that the devices and methods are useful for similar procedures involving the right atrial appendage (RAA), as well as other cardiac and vascular procedures, including atrial incision, atrial volume reduction, ventricular aneurysms, arterial plication for the aortic ectasia or pulmonary artery, cholecystectomy, and numerous other laparoscopic or open procedures to close off vessels, clamp tissues, or any procedures that involve occluding, suturing, resecting, amputating, or stopping blood or fluid flow. Occlusion devices, delivery devices, uses, methods, and related procedures are described in US 2015/0173765, incorporated herein by reference. The present invention provides devices and methods for sealing the region surrounding the tissue puncture site or transfixion points.

In certain aspects, the invention provides a method for occluding an atrial appendage. The method includes deploying an occluder device transfixing a first wall and a second wall of an atrial appendage. In some embodiments, the method involves deploying a plurality of occluders. In some embodiment the method includes deploying at least one occluder element that transfixes a distal wall and at least one occluder element that transfixes a proximal wall, and the occluder elements are clamped together and locked together thereby occluding the hollow structure. In some embodiments, the method includes delivering the occluders via a delivery tube that is a needle or a laparoscopic device. The occluders may be two-part occluders including a distal implant and a proximal implant configured to lock together. The occluder device may be configured to assume a diametrically-reduced configuration for loading into a delivery tube and a diametrically-expanded configuration for deployment adjacent to the atrial appendage.

In embodiments, the method may include encircling the atrial appendage with a sling to lift the atrial appendage away from other heart tissue. In other embodiments the sling is used to support and reinforce to minimize tearing or breaking when the occluder element is deployed through it and the atrial appendage tissue, or project extended closure force on the atrial appendage tissue in the spaces between occluders. The sling can be made of a synthetic polymer or animal tissue and it can include a suture and an adjustment member in a noose configuration. The atrial appendage may be occluded with the sling before deploying the occluder. In some embodiments, the atrial appendage is lifted away from other heart tissue by the sling. A protective device can be placed between the atrial appendage and the other heart tissue or veins and arteries to protect them. In some embodiments, the occluders are placed through one part of the sling, through two walls of the atrial appendage, and through another part of the sling.

In some embodiments, the method also includes amputating the atrial appendage after deployment of the plurality of occluders.

### BRIEF DESCRIPTION OF THE DRAWINGS

FIG. 1 shows an illustration of the human heart.

FIGS. 2A and 2B are cross-sectional and plan views, respectively, of the LAA.

FIGS. 3-5 show an embodiment of the invention using a sling around the LAA.

FIGS. 6-8 show another embodiment of a sling for placement around the LAA.

FIG. 9 shows a device for supporting a tubular structure to be occluded and protecting surrounding tissue.

FIGS. 10 and 11 show devices for delivering multiple occluders to tissue.

FIG. 12 shows a schematic diagram of a two-part occluder for use with the invention.

FIG. 13A shows a needle and a two-part occluder; and FIGS. 13B and 13C show simulated tissue clamped with a two-part occluder with interweaving of the occluder fingers.

FIG. 14 shows an embodiment of an occluder for use with the invention.

FIGS. 15-22 show additional embodiments of the clamping devices for holding an clamping occluder elements together.

FIG. 23 shows an occluder deployed in an atrial appendage.

FIG. 24 illustrates the deployment of sling material around LAA.

FIG. 25 shows an embodiment where the needle is delivered at an angle FIG. 26 shows an embodiment of an occluder.

FIGS. 27-36 show appliers and cartridges for use with the invention.

FIGS. 37-39 show another embodiment of an occluder.

FIG. 40 shows various embodiments of the present invention, with differing levels of finger overlap for single occluder elements in the deployed or relaxed state.

FIG. 41 shows another embodiment of a locking mechanism for the delivery rod with the single piece occluder element.

FIG. 42 shows the delivery steps for the single part occluder, utilizing the locking rod, to deliver and hold the occluder being deployed.

FIG. 43 shows deployment of a delivery tube and rod to reverse occlusion and remove deployed occluder.

FIG. 44 shows further pulling up of delivery rod pulling the proximal occluder fingers into the delivery tube.

FIG. 45 shows an extracted occluder from a tubular structure.

FIGS. 46-49 show embodiments of occluders with a protective sheath.

FIGS. 50-54 show another embodiment of an occluder for use with the invention.

FIG. 54B shows an occluder delivery device.

FIGS. 55-60 show a method of occluding a vessel wherein the vessel is pulled proximally by the distal implant.

FIG. 61 shows a method of occluding a tubular structure

### DETAILED DESCRIPTION

The present invention provides devices and methods for using occlusion devices to exclude an atrial appendage. The invention can be employed during open surgery or thoracoscopy. The invention generally involves placing a row of occlusion clamps at the edge of the left atrial appendage (LAA) to cut off flow from the rest of the heart. The LAA can be simply excluded with the occluders, or it can be subsequently resected.

The invention also provides methods and devices for elevating or isolating the LAA from the rest of the heart before applying the occluders. Elevation can be performed using a laparoscopic device as described herein. It can be aided by using a sling made from a synthetic polymer such as polyethylene terephthalate or PTFE. Alternatively it can be made of animal tissue such as pig or human tissue. The sling can be a noose-like structure that wraps around the base of the LAA and can be tightened with an adjustable tightening member to snugly fit around the LAA. The sling can be various widths, from a narrow suture-like device, to a broad sheet up to several centimeters in width. In embodiments using broader slings, the occluder devices can be placed through a sling that has been wrapped around the LAA.

Occlusion devices for use with the present invention are described in U.S. patent application Ser. No. 13/348,416, filed Jan. 11, 2012; U.S. patent application Ser. No. 13/857,424, filed Apr. 5, 2013; and U.S. patent application Ser. No. 14/272,304, filed May 7, 2014, each of which is incorporated by reference herein in its entirety. The delivery device can be a hollow needle or a laparoscopic device. The delivery device can be configured to contain one or more occluders. Delivery devices for use with the present invention are described, for example, in U.S. patent application Ser. No. 14/639,814, filed Mar. 5, 2015 (published as US 2015/0173765) and U.S. patent application Ser. No. 15/226,577, filed Aug. 2, 2016, incorporated by reference herein in their entirety. Occluders may have a polymeric or organic coating to prevent rubbing against surrounding tissue and causing erosion or damage.

Throughout this disclosure, the terms “tubular structure” and “vessel” are used interchangeably to mean anatomical structures that can be occluded, including the LAA. It will be appreciated, however, that the methods of the present invention can be applied to occlusion of structures other than the LAA.

Atrial Appendage Procedures

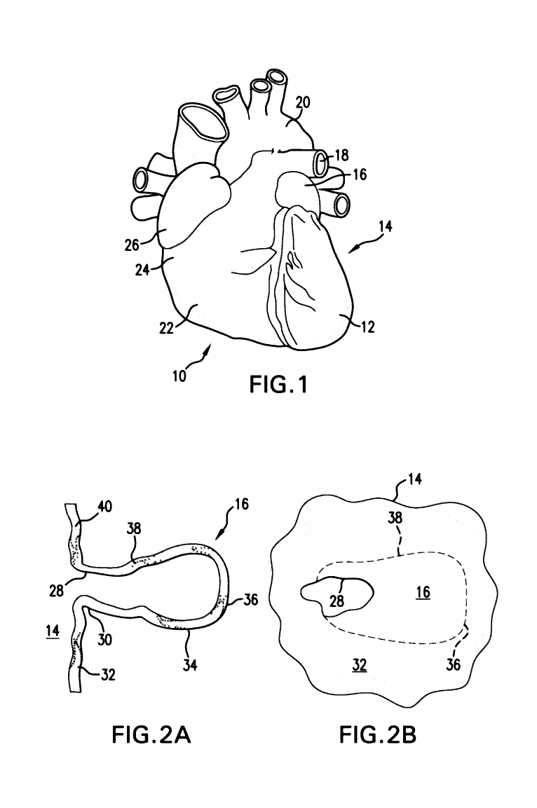

Referring to FIG. 1, heart 10 is illustrated to show certain portions including left ventricle 12, left atrium 14, left atrial appendage (LAA) 16, pulmonary artery 18, the aorta 20, the right ventricle 22, the right atrium 24, and the right atrial appendage 26. As is understood in the art, the left atrium 14 is located above the left ventricle 12 and the two are separated by the mitral valve (not illustrated). The LAA 16 is normally in fluid and electrical communication with the left atrium 14 such that blood flows in and out of the LAA, and electrical impulses conduct to and from the LAA 16 as the heart 10 beats.

FIGS. 2A and 2B are a schematic cross section of LAA 16 and a plan view of the ostium to the LAA. The chamber of the left atrium 14 and the interior of LAA 16 are shown in communication via ostium 28. The LAA is further defined as having base portion 30 where it attaches to pericardial surface 32 of the left atrium 14, and body portion 34 distal to the point of attachment of LAA 16 with the left atrium, including apex 36. Walls 38 of LAA 16 are vascularized heart tissue substantially similar to the walls 40 of the left atrium. As shown in FIG. 2B, ostium 28 may have an irregular circumference, and body portion 34 of the LAA may extend from the left atrium at a shallow angle, making it difficult to implant a circular occlusive member within the LAA.

Occluding or resecting the LAA according to the present disclosure involves placing one or more occlusion clamps along the LAA to cut off flow from the rest of the heart. Various methods and devices for performing such a procedure are described herein.

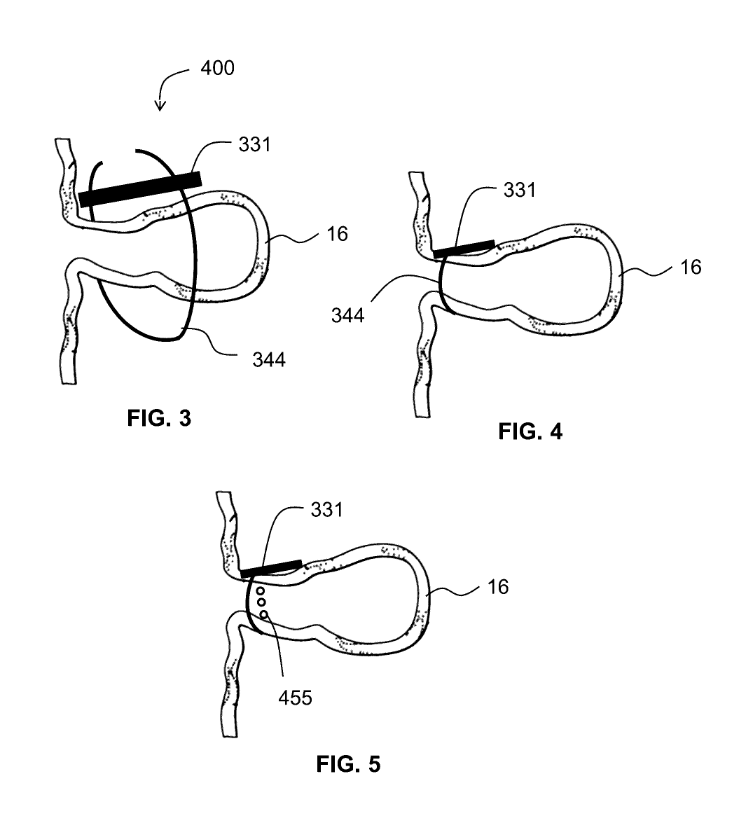

Optionally a sling can be used in conjunction with the occluders. The sling may help to elevate the LAA away from the heart or it may combine with the occluders to provide additional pressure. The occluders, however, are configured to occlude the LAA with or without the sling. FIGS. 3-5 show the use of an optional sling in accordance with the present invention. The sling can be narrow or broad. The embodiment shown in FIGS. 3-5 is narrow, but a broader sling can be used for the described methods as well (additionally, broad slings are described below in FIGS. 6-8. The sling 400 can be tied around the LAA 16 to lift it away or elevate it from the rest of the heart tissue. The sling 400 can isolate the LAA 16 from the heart during a procedure to prevent puncture of the heart tissue with an occluder delivery device. The sling 400 can be tightened around the LAA 16 using a suture 344 and the adjustable member 331. The suture 344 can optionally be secured to the LAA 16 on one side (via suturing) and adjustable on the other side (via the adjustable member 331). The adjustable member 331 may be a piece of plastic, polymer, or other biocompatible material. It can be made of the same material as the suture material. The suture 344 can be threaded through or into the adjustable member 331 and pulled through it to tighten around the appendage 16. The suture 344 forms a noose-like structure that applies a pressure around the LAA 16.

The sling 400 allows the LAA 16 to be lifted up and manipulated. Upon tightening (FIG. 4), the sling 400 can close off the LAA 16 from the rest of the heart to remove the blood flow from the LAA 16. The sling 400 can also provide support to the fragile tissue of the LAA 16. The sling 400 can be cut to the size and shape of the LAA 16.

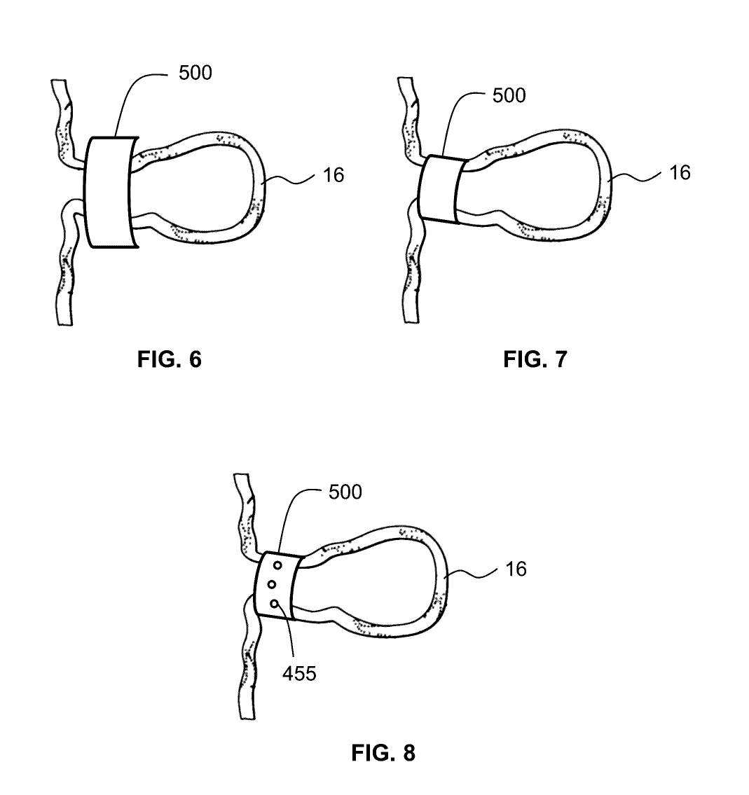

Once the sling 400 is tightened, occlusion clamps 455 can be inserted through the LAA to transfix opposing walls of the LAA, as shown in FIG. 5. The tightened sling 400 can reduce the number of occluders 455 that are required. Once the occluders are in place, the sling can be removed or it can remain in place. Generally, the length of occlusion required is approximately 3-4 cm.

Occlusion can be the final step of the procedure, or in some cases, the LAA 16 may be amputated. The LAA 16 can be amputated at or near the position of the occluders. Occlusion may be performed with or without the sling described above. The procedure can be performed on a beating or non-beating heart.

The invention contemplates other embodiments of the sling as well. As discussed above, the LAA is made of fragile tissue, which may be damaged by traditional methods of suturing or stapling, leading to excessive bleeding. The sling of the present invention can be a broader piece of material, which can compress a wider area of the LAA through which the occluders can be inserted. The compression supports the area around where the occluders penetrate the LAA tissue and prevents bleeding.

Accordingly, FIGS. 6-8 show an embodiment where the LAA is occluded with occluders placed through the sling rather than next to it. In FIG. 6 a broad sling 500 is placed around the LAA. The sling 500 can have a width of up to several centimeters. It can be one or more layers of material. The width can be adjusted by a user, for example by adjusting the layers relative to one another or by folding the material. The sling 500 can be tightened around the LAA 16 in a manner similar to the embodiment of FIGS. 3-5. In some embodiments, the sling 500 can be shaped by the surgeon to fit snugly around the LAA 16 in the location where occlusion is desired.

FIG. 7 shows the sling 500 tightened around the LAA. The broad sling 500 adds support to the LAA in the location to be occluded, i.e., the occlusion line. FIG. 8 shows several occluders 455 having transfixed the sling 500 and the LAA 16. Having width to the sling 500 allows the needle delivering the occluders 455 to pass through the one portion of the sling, the two walls of the LAA, and finally a second portion of the sling. Penetrating the sling that has been wrapped around the LAA adds strength and prevents the LAA from tearing. Additionally, it extends the area of compression and so the procedure may require fewer occluders to be used to complete the occlusion line. The line of occluders is generally a length of around 3-4 centimeters. The extended area of compression provided by the sling reduces the amount of strain on the tissue in the immediate vicinity of the occluders. With prior art devices such as staples and sutures, the location that the LAA is punctured is susceptible to tearing or bleeding. The present invention provides a device that puts a more uniform pressure on the surface of the LAA, making it less likely to tear at the occlusion sites.

The sling can be shaped (for example, at the time of surgery by the surgeon) to conform to the edge of the LAA. By doing so, the occlusion line can be accurately defined. That helps ensure that the entire LAA is removed, and prevents leaving part of the appendage, which may result in clot formation and distal emboli.

Various accessory instruments are contemplated for use with the present invention. For example, a retractor can be used in conjunction with a guard placed on the left ventricle under the LAA to prevent misplacement of the penetrating needle during delivery of occluding elements. Such protective devices and methods are described in more detail below. In certain embodiments, the retractor may be solid or may have an adjustable width. It may include a reflective surface (such as a mirror) so that needle penetration and distal occluder clip deployment can be observed.

Lifting the LAA 16 away from the heart allows a metal plate, or mirror or mirror like structure, or other protector to be placed between the area to be occluded and the rest of the heart. Various devices are disclosed that are useful for protecting surrounding tissue from that which is to be occluded.

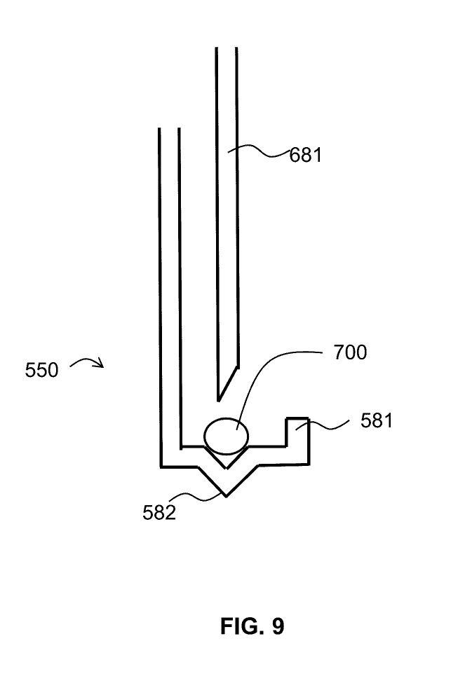

Sling devices of the present invention can be used in conjunction with protector devices that shield non-target tissue from the occluders and occluder delivery devices. For example, a sling can be placed on the LAA and then used to pull the LAA away from the heart, so that a protective device can be placed underneath. Various devices for protecting surrounding tissue are disclosed below, and with reference to FIGS. 9-16.

FIG. 9 shows an embodiment of a laparoscopic device for supporting a delivering an occluder to a structure 700 to be occluded. The device includes a stop protector 550 with a trough 582 for supporting a vessel 700. The stop protector 550 also includes a lip 581 to prevent the vessel 700 from sliding off of the device. The stop protector 550 supports the vessel 700 while a needle 681 delivers an occlusion device (not shown). The needle 681 and the stop protector 550 can be part of the same device, or they can be separate devices. The needle 681 can be any needle or delivery tube described herein.

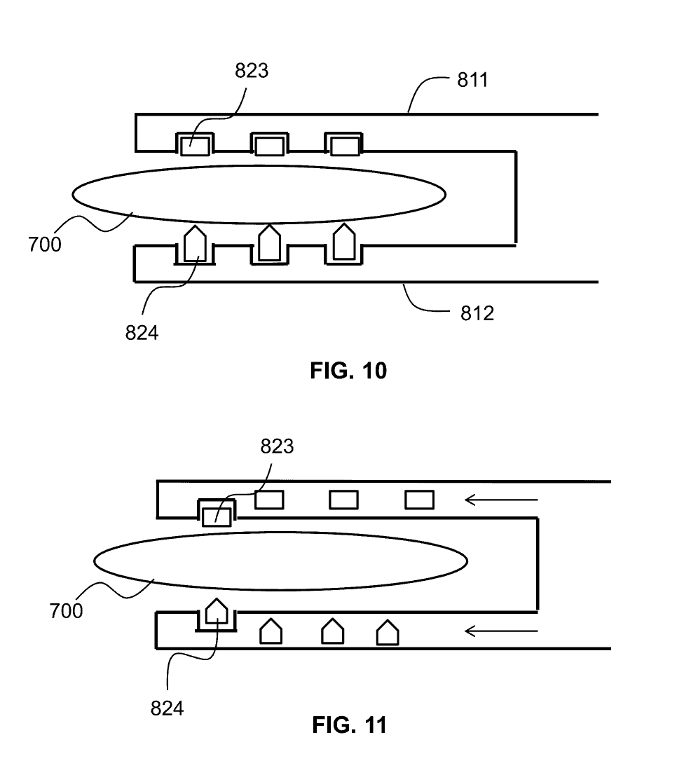

FIGS. 10 and 11 show another embodiment of a device for delivering occluders while protecting surrounding tissue. The devices shown in FIGS. 10 and 11 resemble an alligator's “jaws.” The top jaw 811 is configured to deploy several female occluder elements 823 and the bottom jaw 812 is configured to deploy several corresponding male occluder elements 824. The jaws are aligned so that the male and female occluder elements can come together to occlude a tissue 700, which may be an LAA, therebetween. In operation, the jaws can move towards each other while remaining parallel, such that when the jaws come together, the several occluders are deployed into the tissue 700. When the jaws 811 and 812 activate, the tissue 700 therebetween is compressed and occluded.

In the embodiment shown in FIG. 11, each jaw has only one occluder deployment location, and several occluder elements are disposed within each jaw. The occluder elements can be spring loaded so that when one occluder is deployed, the next occluder takes its place in the chamber. The occluder elements move in the direction of the arrows shown. The action of this embodiment of the device is similar to the operation of a stapler.

By deploying occluders with the jaw-like devices of FIGS. 10 and 11, only the desired tissue is transfixed by the occluder, and surrounding tissue is protected.

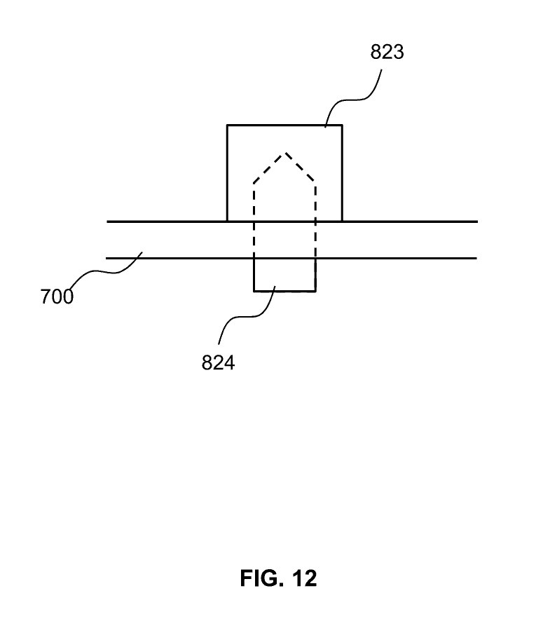

FIG. 12 shows a schematic drawing of a two-part transfixing occluder for use with the invention. The male element 824 transfixes the tissue 700 and is received by the female element 823. The two occlusion elements lock together to occlude the tissue therebetween. Other two-piece occluders for use with the invention are described in U.S. Patent Publication 2014/0243857, incorporated by reference.

Other devices and methods for protecting surrounding tissue are described in U.S. Patent Publication 2015/0173765, incorporated by reference.

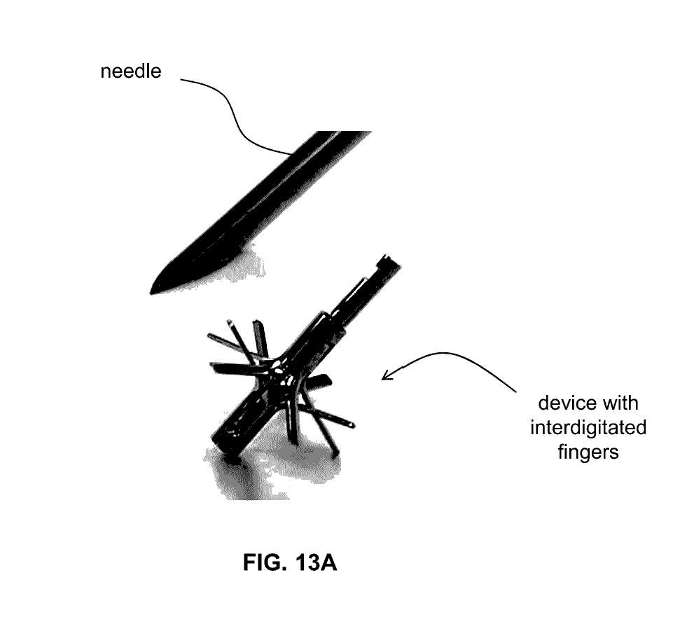

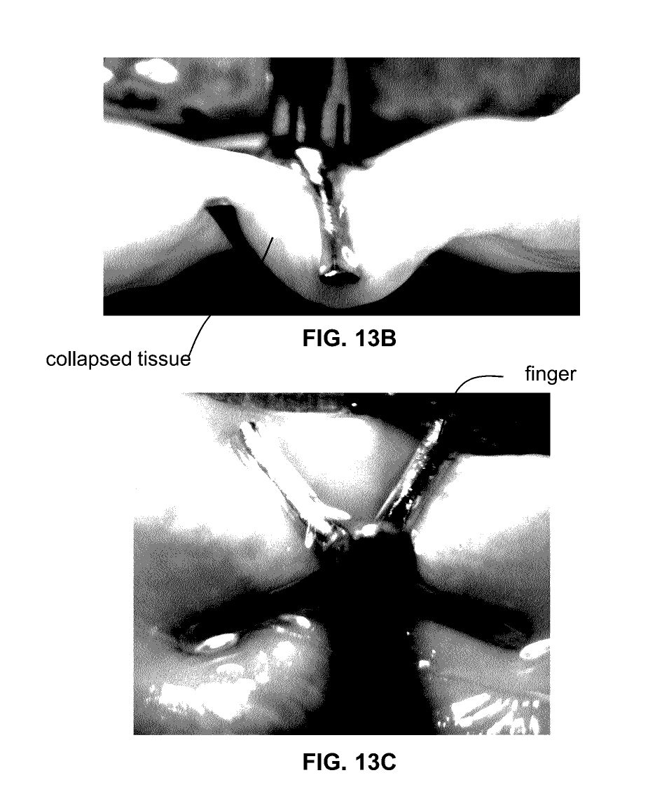

FIG. 13A shows an 18 gauge needle and interdigitated fingers of a two-part occluder. Single part occluders with a similar interdigitation of the proximal and distal fingers are also envisioned in embodiments of the invention. A benefit of interdigitation of the fingers is that structures can be compressed between the fingers without necrosis of the tissue being clamped. This helps prevent tearing and necrosis and minimize leakages when treating cardiac tissue.

FIGS. 13B and 13C show close-up views of simulated tissue clamped between the interdigitated fingers, producing an interweaving of the collapsed tissue. Due to the interdigitation of the fingers, the region around the transfixion hole produced by the needle when deploying the occluder, is sealed and prevents any blood or bile or other leakage from flowing out of it. This is crucial in the case of the LAA, as one of the key problems with current occluders, staples or clamps for clamping the LAA is that they can cause bleeding when deployed, and they do not have a pressure zone surrounding the point where they pierce the tissue, and as a result can lead to uncontrolled bleeding when the patient is taken off the heart-lung bypass machine. Uncontrolled bleeding will require that the patient be put back on the heart-lung machine, which is a very risky procedure. As such, there is a very strong desire to avoid any bleeding from the occlusion sites once the procedure is completed and the patient is removed from the heart bypass machine. FIG. 13B shows a side-view of the interweaving of the occluder fingers, and FIG. 13C shows a top view of the interweaving of the occluder fingers for the proximal occluder element.

The circular interdigitation of the occluder fingers securely brings the tissue together and provides a complete hemostatic area. The flexibility of the fingers allows the occluders to accommodate various tissue thicknesses. The occluders create an area of compression that extends beyond the perimeter created by the fingers, thus allowing them to be placed with a space between them, thus requiring fewer occluders to complete the suture line.

Importantly, because the occluders can each be placed at a location of the clinician's choosing, they do not have to conform to a linear path along the LAA or other structure to be closed off. The occluders can be used to create a non-linear line of occlusion. This is especially important for atrial appendage procedures because the orifice of the atrial appendage is non-linear. The occluders are thus more effective than linear clamps known in the prior art. Allowing the occluders to be placed non-linearly along the orifice of the LAA prevents the creation of residual recesses where a clot can form. Most current devices for LAA failed because they are clamp-like structures that cannot follow the shape of the orifice and thus leave recesses of the LAA and do not prevent later embolization.

The occluders alone are capable of closing off the LAA without the use the sling described above. In some embodiments, it may be desirable to eliminate the sling, which reduces the thickness of the area of occlusion and allows the occluders to interdigitate better. In other embodiments, the sling may be desirable.

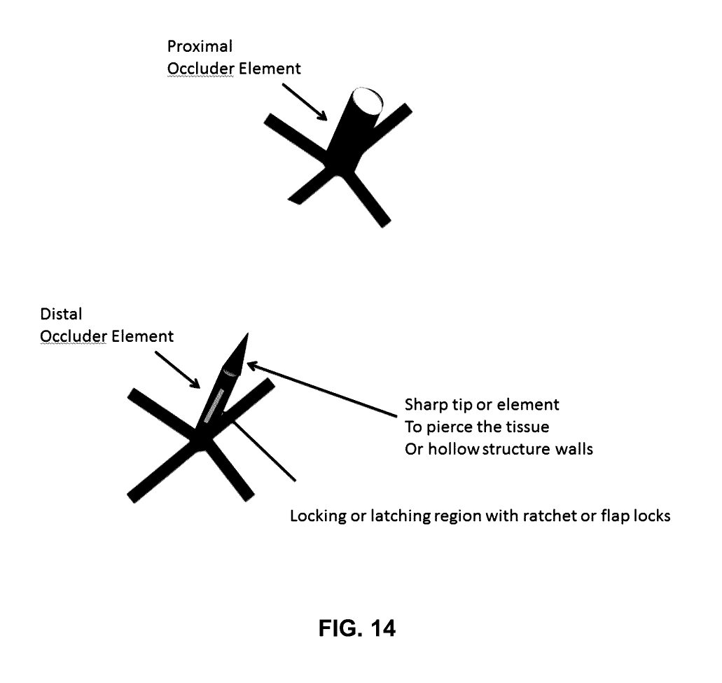

FIG. 14 shows an embodiment of a two part occluder prior to occlusion of a hollow structure, with a distal element that has a sharp element to pierce the LAA and a locking mechanism. When proximal and distal occluder elements are pushed or clamped together, the locking mechanism snaps or locks the occluder elements together. Note, in other embodiments the proximal occluder may have a sharp tip to pierce and occlude the hollow structure, or both elements may have these features. The distal occluder element penetrates or pierces the distal wall of the hollow structure, and the proximal and distal occluder elements are clamped and locked together compressing hollow structure. In this embodiment the distal occluder element pierces both distal and proximal walls of hollow structure.

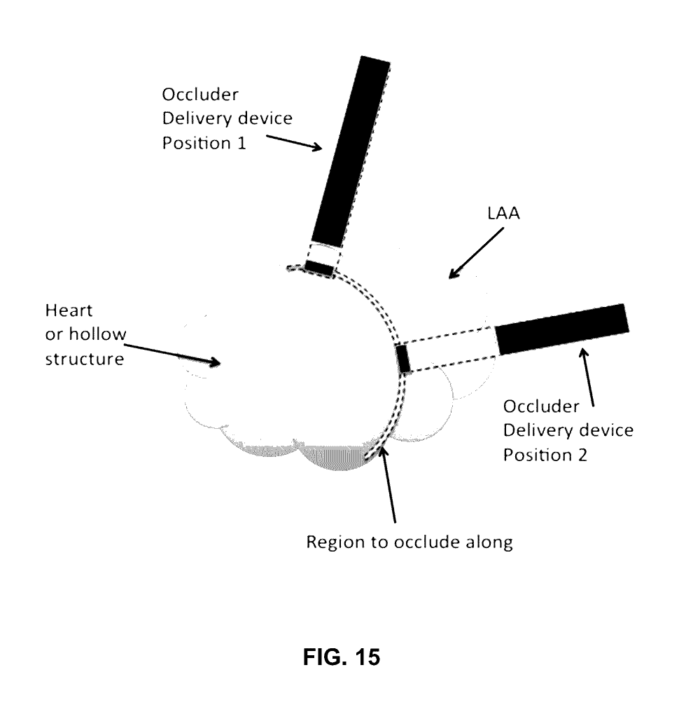

FIGS. 15-23 show additional embodiments of the clamping devices for holding an clamping occluder elements together.

FIG. 15 shows a top view of a heart including the LAA. A clamping occluder delivery device is shown at two different deployment positions. Note that the length of the clamping jaws is user controllable and adjustable so that they can easily adjust to the curvature and specific structure of the hollow structure to be occluded.

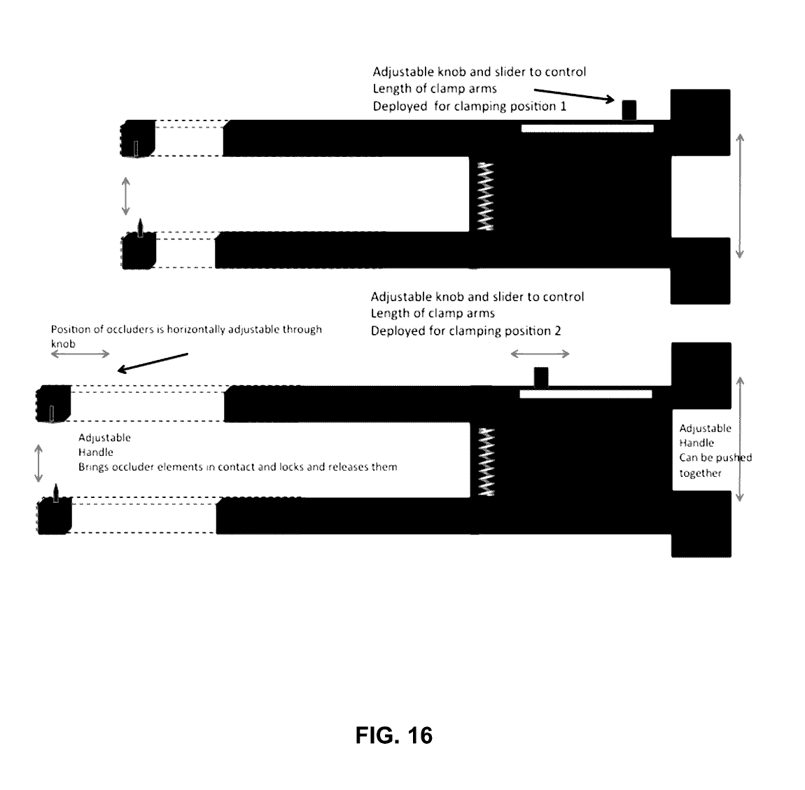

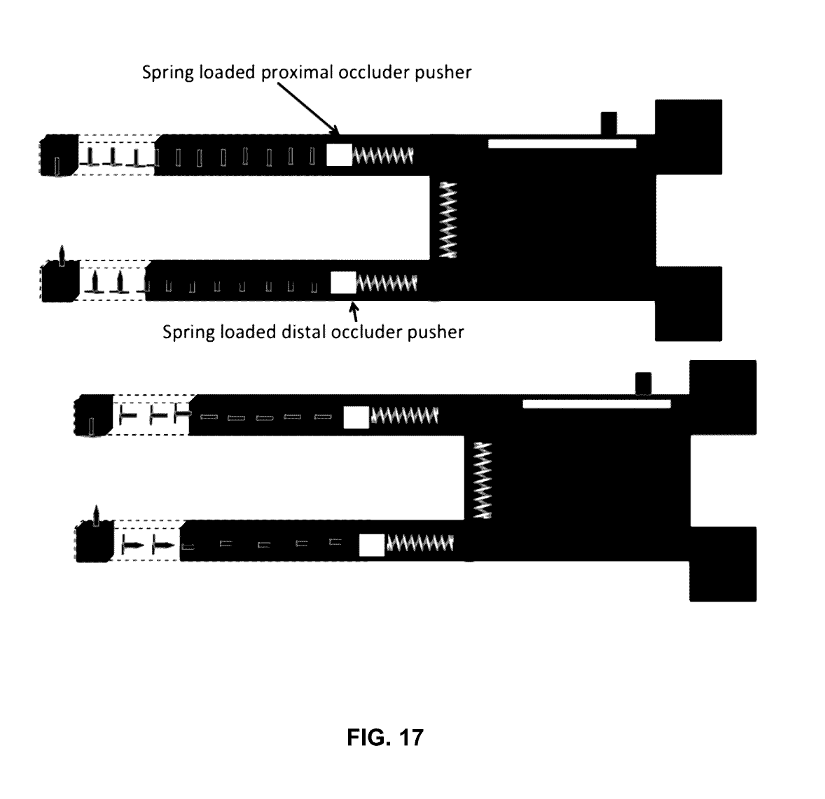

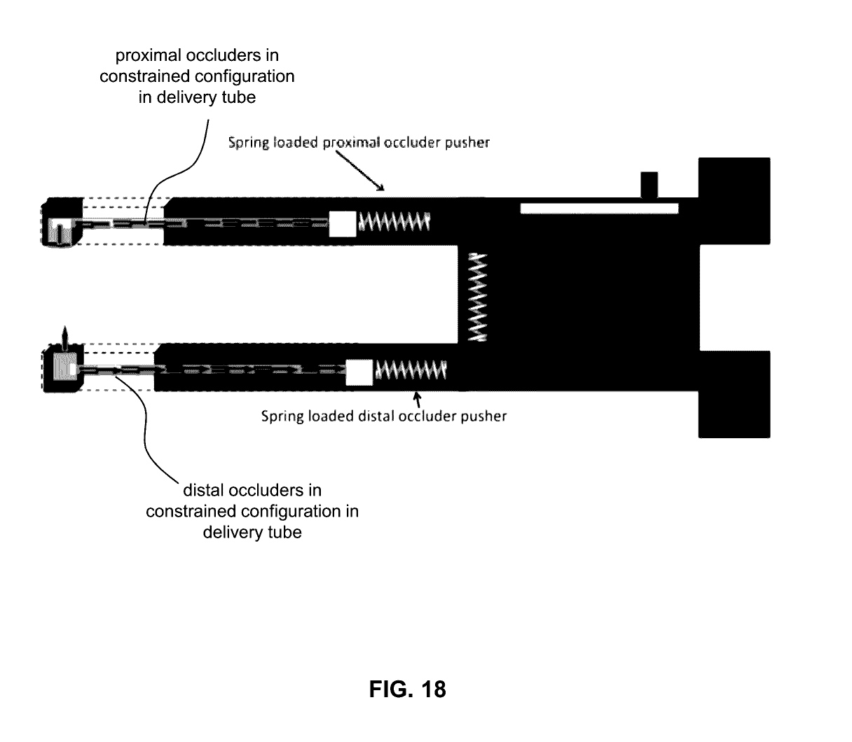

FIG. 16 shows a side view of an occluder delivery device with adjustable clamping jaws. FIG. 17 shows delivery devices accommodating multiple occluding elements, loaded into the occluder delivery device in various configurations. Once the proximal and distal occluder elements have been ejected across a hollow structure, or multiple layers of materials such as tissue or polymers, the spring pushing elements move the next pair of occlusion elements to a position where they are ready to be clamped together. In some embodiments of the devices shown, the fingers of the occlusion elements are unconstrained in the delivery tube and are fully opened, however, in other embodiments, such as in FIG. 18, they may be constrained in a delivery tube, and only open up when ready to be deployed.

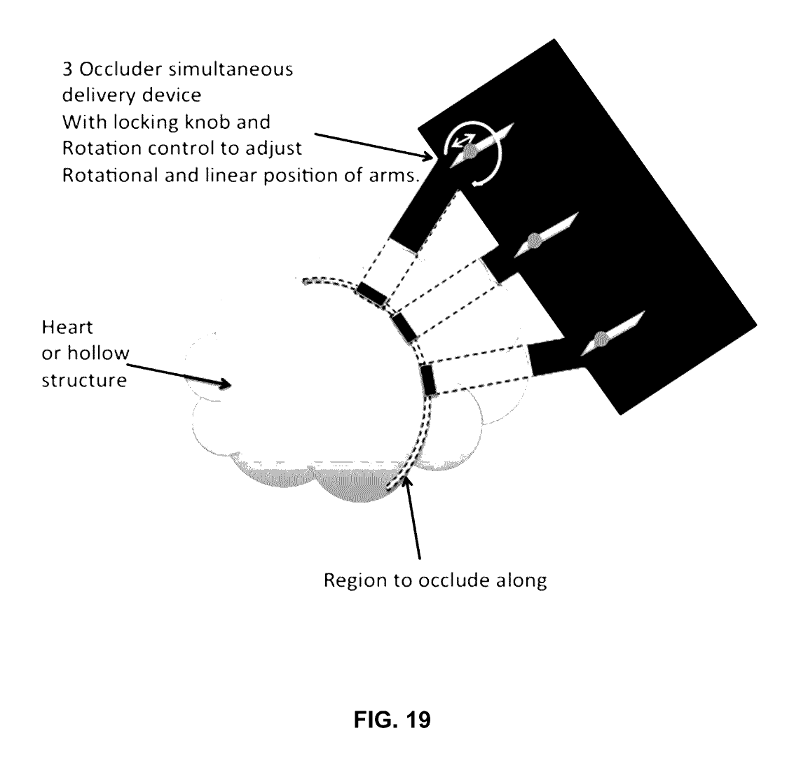

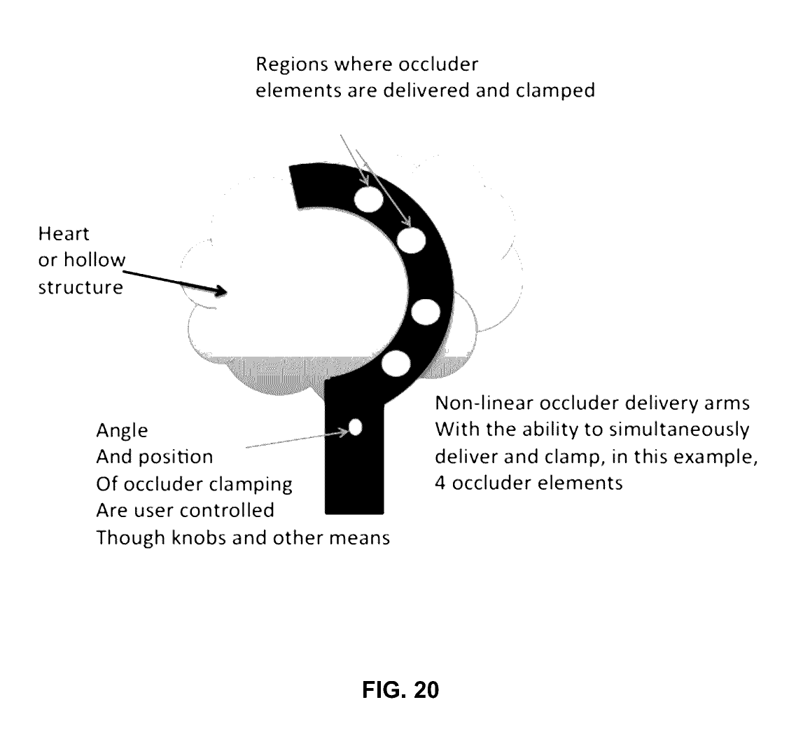

FIG. 19 shows a delivery device capable of deploying three occluders simultaneously while customizing the occluder arms lengths and angular positions prior to the occlusion utilizing linear and rotating locking knobs. FIG. 20 shows curved clamping elements capable of delivering multiple occluder elements simultaneously in a non-linear configuration. The angle, curvature and spacing of the regions for occlusion can be adjusted by the user, by activating various knobs.

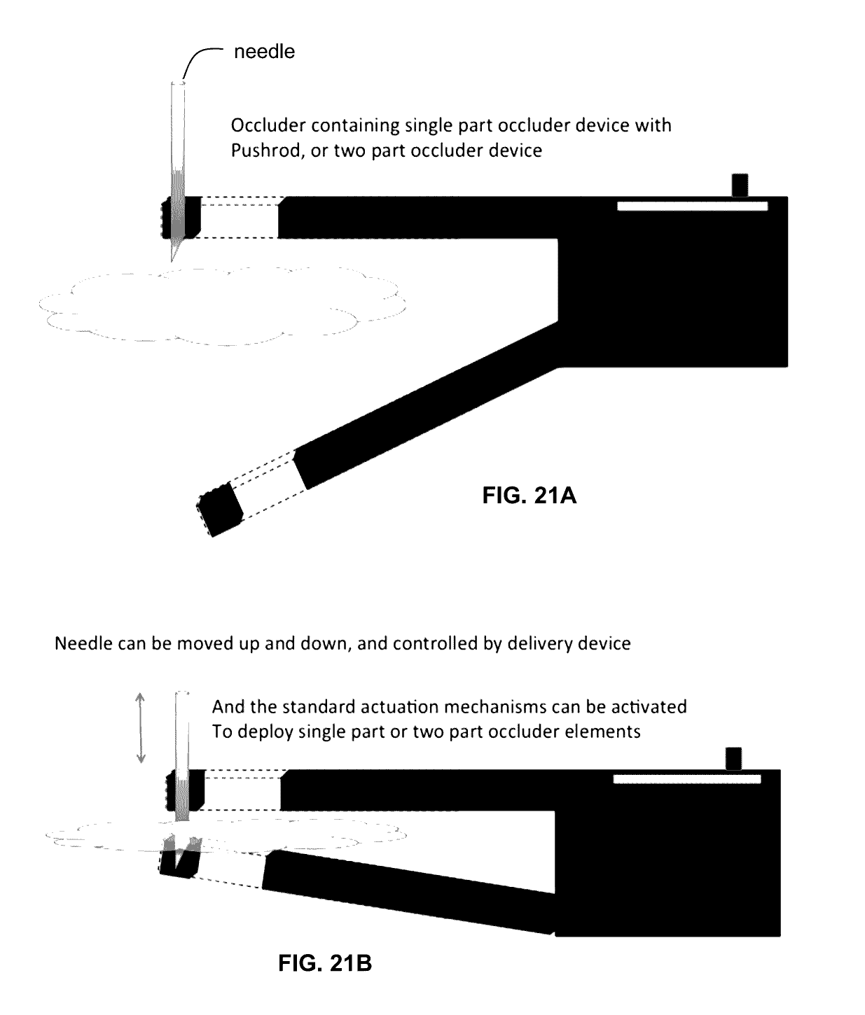

FIG. 21A shows an occlusion delivery device containing a needle. The clamping arms grip and clamp the tissue together at the desired location. Next the occluder delivery needle delivers either a single part occluder or a two part occluder through the hollow structure or tissue or material layers, and clamps them together to produce an occlusion or secure clamping. FIG. 21B shows clamping the delivery device arms and piercing of hollow structure walls to deploy the occluder.

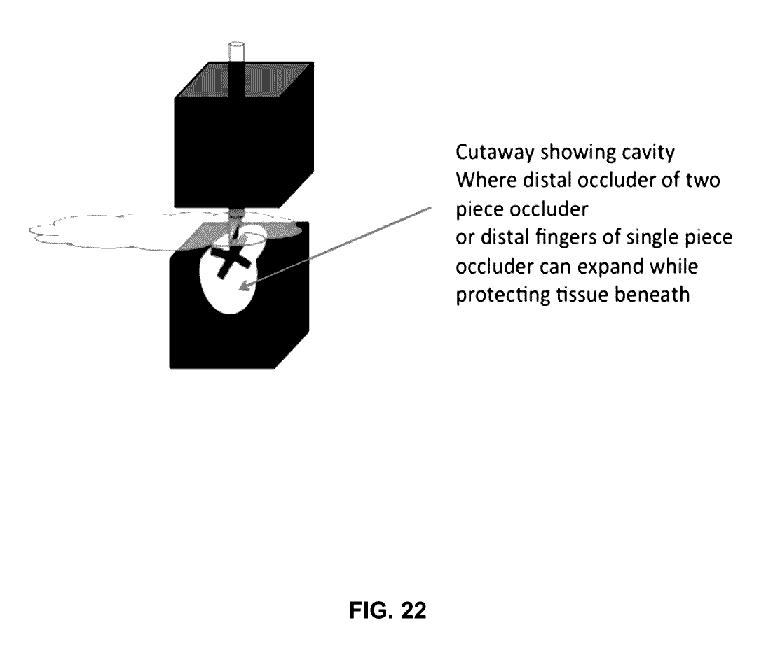

FIG. 22 shows an enlarged and cutaway view of the arms of a clamping device. The cavity is visible that allows expansion of the distal occluder element beneath the tissue to be occluded or clamped.

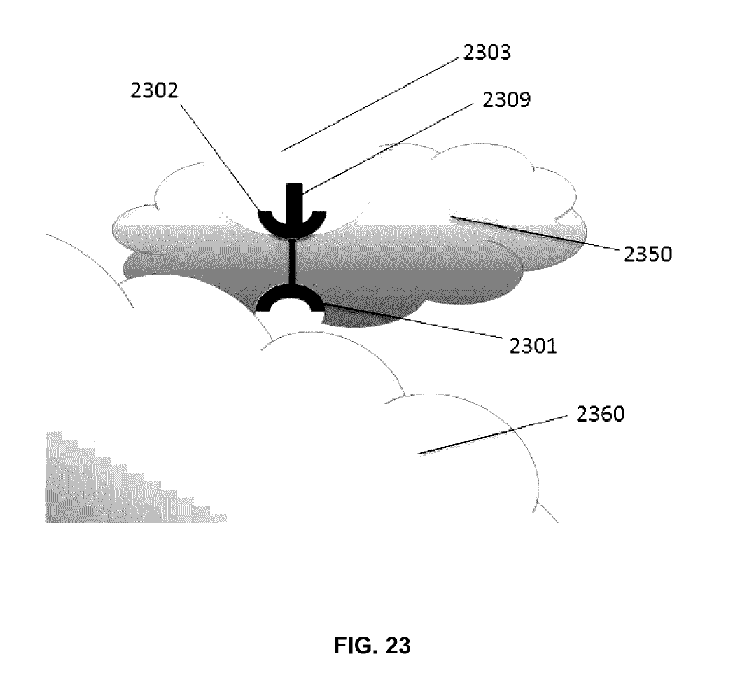

FIG. 23 show proximal and distal occluder elements locked together such that the distal occluder element 2301 forms a concave region in the atrial appendage 2350, and is thus embedded in a way that the occluder element is fully contained in the atrial tissue and does not project beyond the atrial tissue. That way, the occluder element does not contact, rub against, or erode cardiac tissue or blood vessels in or near the heart 2360. The proximal occluder element 2302 may have a protective polymeric coating 2303 that further protects surrounding tissue. The proximal element 2302 connects with the distal element 2301 by the locking element 2309, which extends through the appendage 2350. Although not shown in FIG. 23, as discussed herein, the proximal and distal occluder fingers are designed to interdigitate. The interdigitation creates a repeated undulation in the tissue that is clamped.

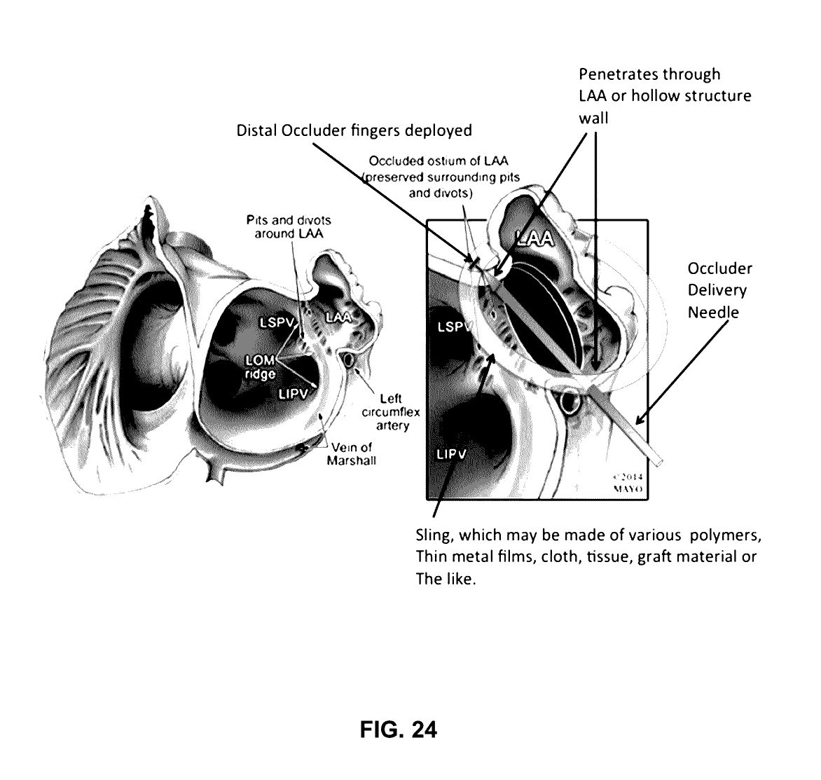

FIG. 24 illustrates the deployment of sling material around LAA and thorough which the occluder is deployed in the present embodiment to enhance and reinforce the LAA tissue integrity, and minimize abrasion to the LAA tissue. In the present embodiment, the delivery needle for the occluder is deployed through the sling. Other clamping devices disclosed herein can also be delivered through the sling.

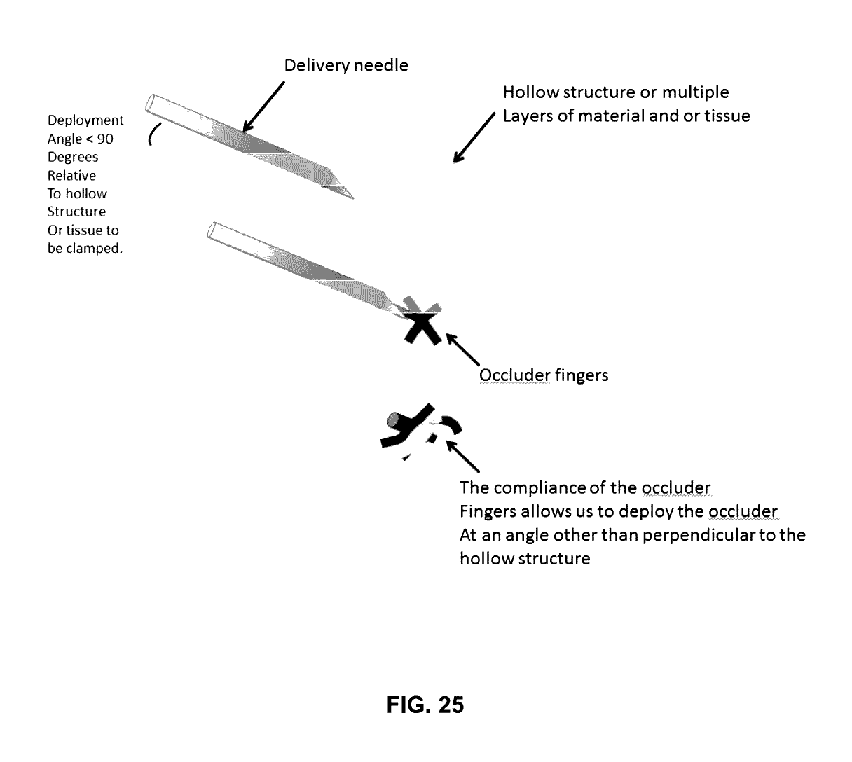

FIG. 25 shows an embodiment where the needle is delivered at an angle. This allows the occluder to lay quite flat. In some embodiments the occluder has a very low profile and can deliver at 90 degrees, while still remaining quite flat.

In an embodiment, devices and methods of the invention are useful for occlusion of tubular structures with polymeric occlusion elements that are resorbable. The occlusion elements can resorb over time, leaving the downstream tissue fused, but no residual occluder element.

In other embodiments, the occluder can be used in conjunction with laser or RF ablation for treatment of various conditions including venous reflux. By occluding the vessel with our occluder upstream, e.g., at the Sapheno-femoral junction, the occluder acts to deflate the vessel, as it stops blood flow into the vessel, collapsing the vessel, and as a result reduces the separation between the walls of the vessel and the laser or RF probes which in turn, reduces the amount of energy needed to be deployed by laser or RF ablation, and as a consequence can reduce or eliminate the need for tumescent anesthetic deployment surrounding the vessel. The tumescent step is a time consuming, uncomfortable step for the doctor and patient. Also, reducing the amount of energy imparted by the laser or RF probe can reduce the side effects to the patient and avoid the risk of burning the vessel tissue.

Another delivery device for delivering the disclosed occluders for any laparoscopic or open procedure is shown in FIGS. 26-36. A key aspect of the device is that one side of the occluding device has a protective structure 2615 (as shown in FIG. 26), which may be a plate, or bulbous material made of a metal or plastic and may in some embodiments be coated with a polymer or entirely made of a polymer or tissue material. The purpose of this design is to protect the tissue on the side to which the piercing element is being driven. In the cardiac case, the protective element may rest against the heart. Protective elements may be on both sides of the device, protecting surrounding tissues form the occlusion elements.

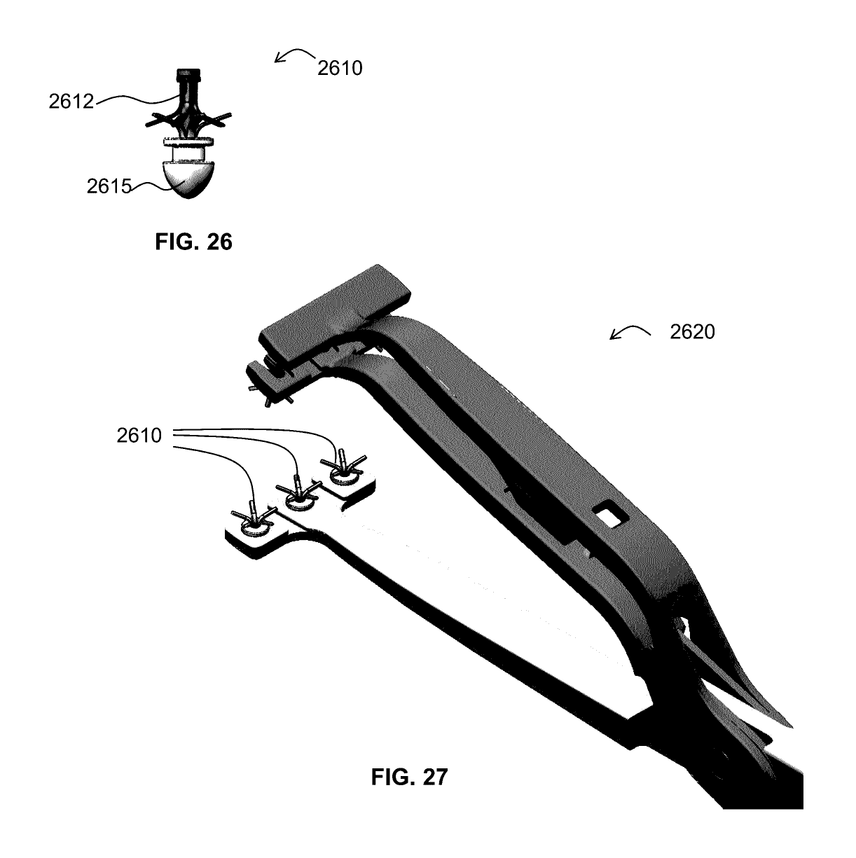

The delivery device 2620 can be configured to deliver any of the two-piece occluders disclosed herein to transfix opposing walls of heart chambers or vessels. As shown in FIG. 27, the delivery device 2620 allows a selective number of clips 2610 (e.g., up to 3 occlusion clips) to be loaded and delivered at one time. This allows a curved or straight occlusion configuration to be performed, which is important with LAA amputation where the entire LLA needs to be amputated to prevent pockets of residual clot forming which can occur with a straight configuration. In embodiments, the occlusion elements 2610 may be various sizes and have various locking stations, allowing for variable thickness of tissues to be sealed together. The occluding elements 2610 may be made of Nitinol or some other flexible material with a locking central rod of titanium or some other rigid material. The inter-digitation of the clip 2610 provides for tension along the suture line (clip line) and in-folding of the tissues such a to allow the two edges of the suture line to coapt and allow the occluders to be placed a some distance from each other so that the occluders do not have to be in contact with one another to achieve sealing of the suture line and avoid any problems with bleeding.

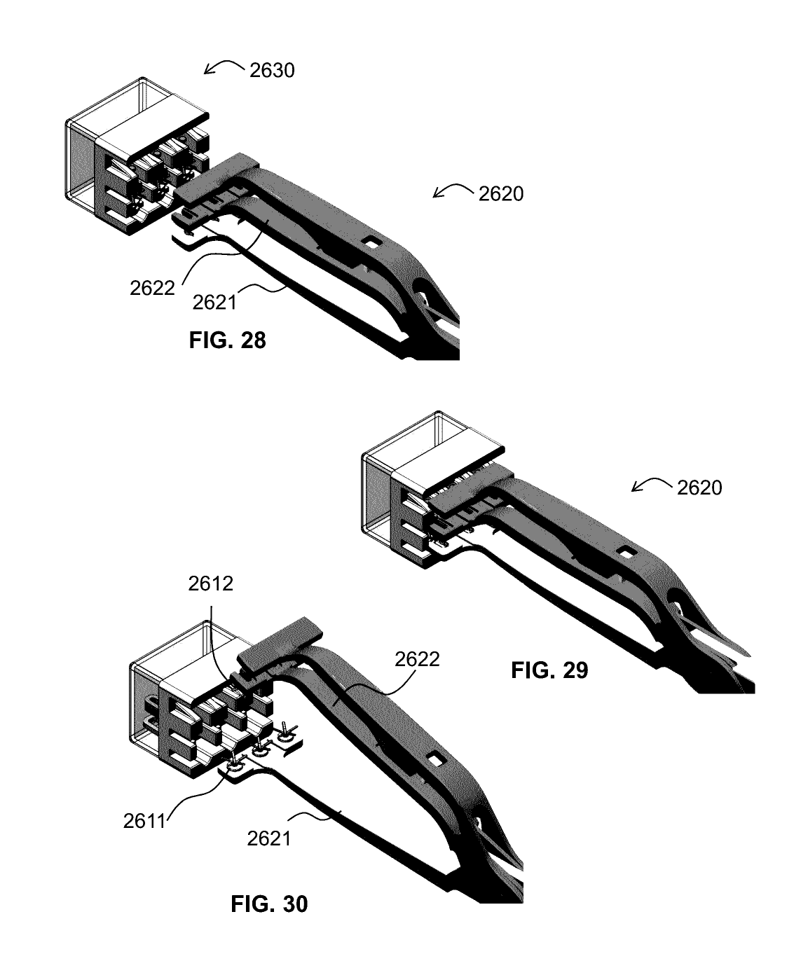

The delivery system is shown in FIGS. 28-36. In general, the system includes the occluder elements, the occluder element cartridge, and the occluder applier.

The occluder elements can be any that have been described herein or in related publication US 2015/0173765. The occluder elements can be loaded into the two jaws 2621 and 2622 of the device in their preformed shapes, as shown in FIGS. 28-30. In some embodiments, the locking mechanism between the two occluders is a sharpened rod which will transfix both sides of the tissues. This rod locks the two elements together and allows secure occlusion of the wound edges or areas that need to be coated and sealed together. The end of central portion of the distal element may be configured to cover the tip of the sharpened transfixing rod. The occluder rod can have adjustable or multiple locking stations or elements, so that depending on local thickness of the tissue, the occlusion elements will be locked at a specific separation or distance from each other.

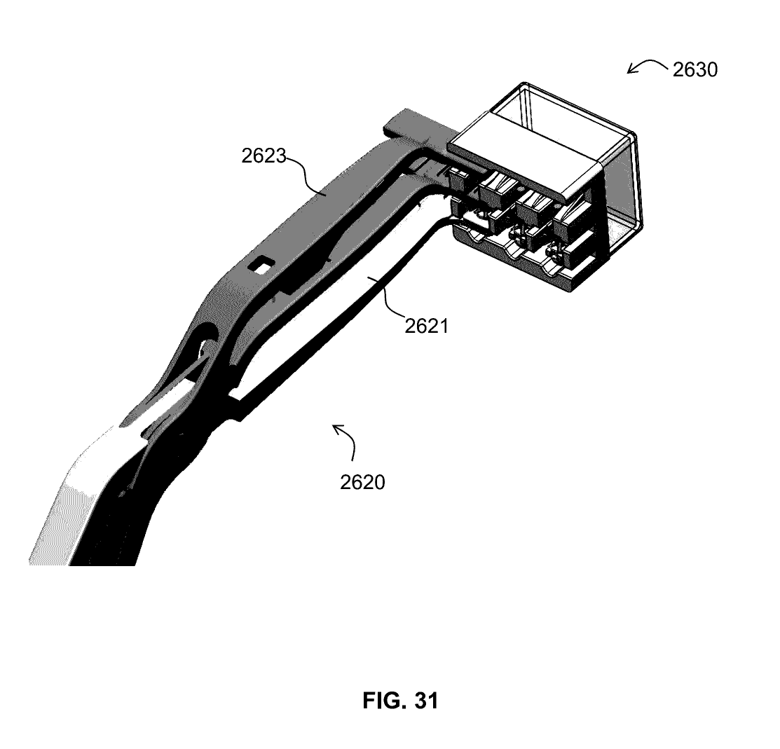

The occluder element cartridge 2630, as seen in FIG. 28-30, provides a designated number of occluders, e.g., 3-12 occluders. It provides the ability to load a single occluder (see FIG. 31) or up to 3 occluding elements at one time into the occluder applier 2620 (delivery device).

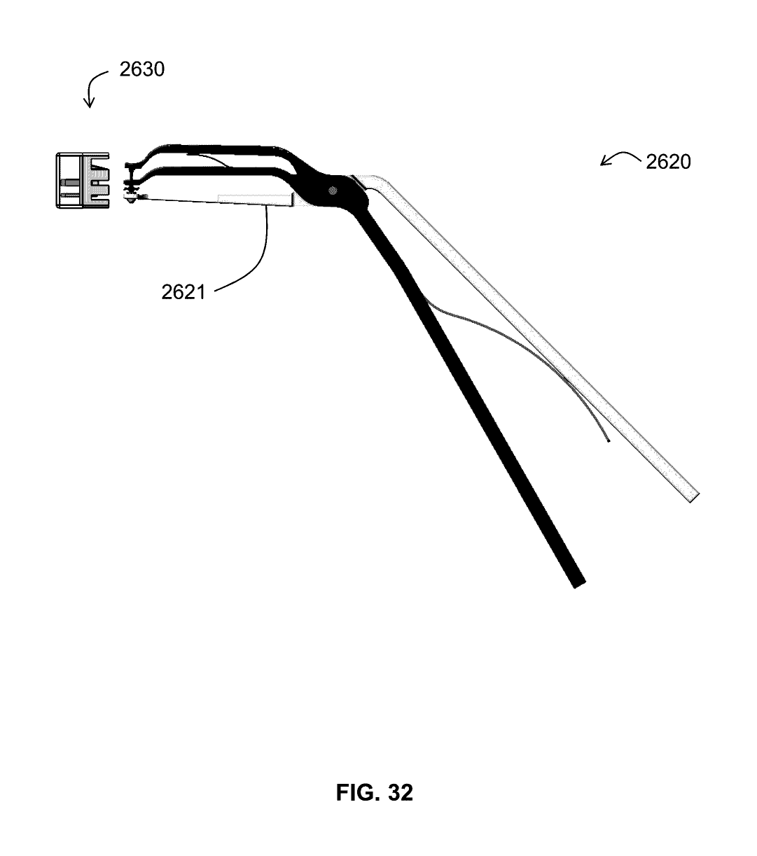

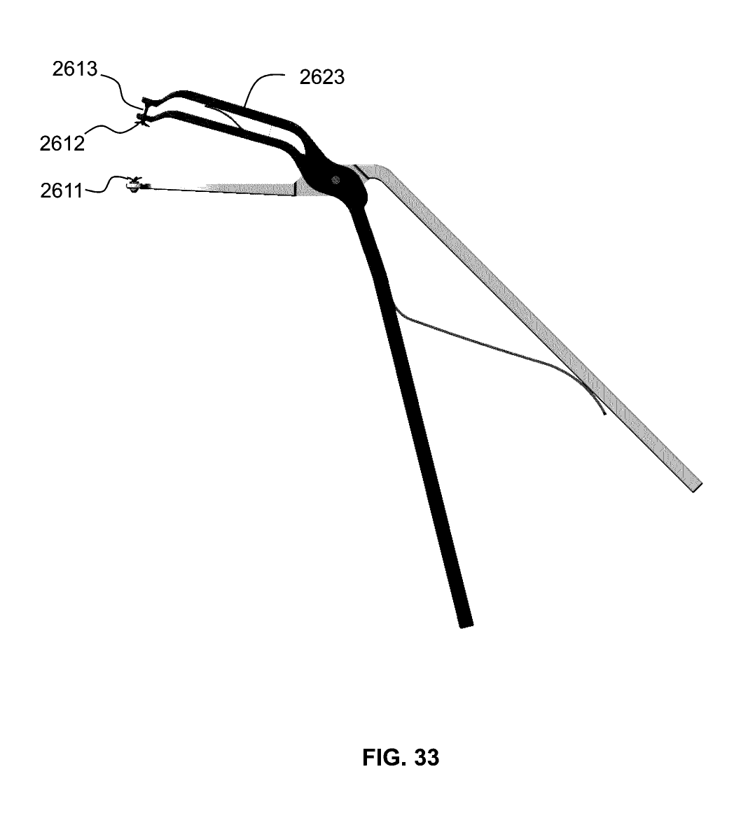

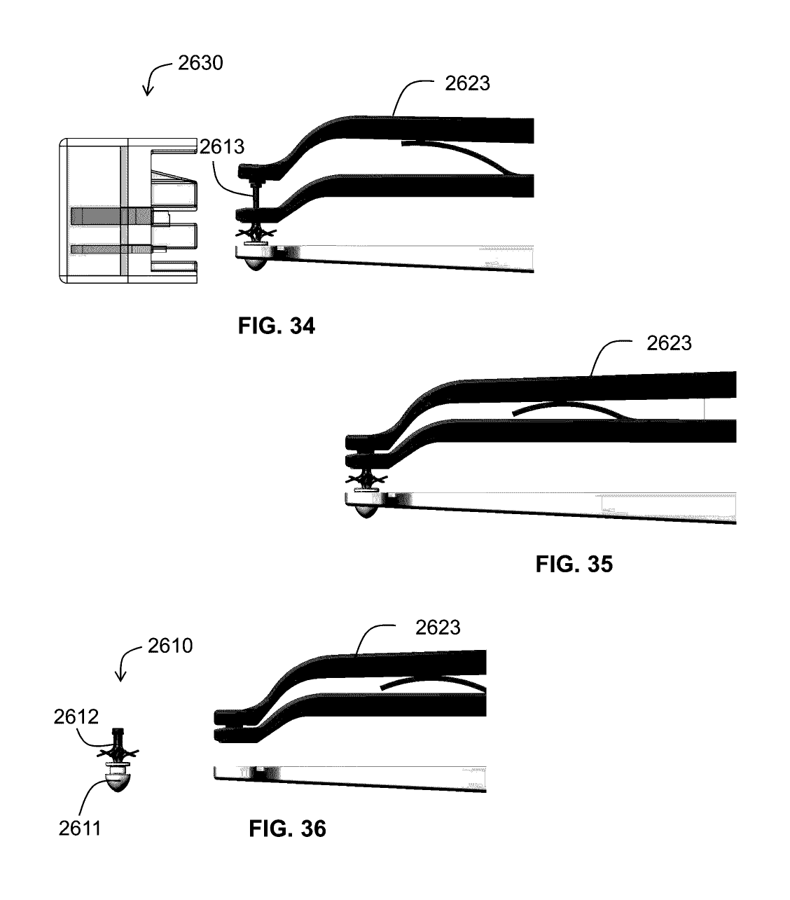

The delivery device 2620, also referred to as the occluder applier, is a clamp-like device (see FIG. 32) with two jaws 2621 and 2622 where the clips can be loaded from the cartridge 2630. The jaws 2621 and 2622 can expand to accommodate the size of an object to be occluded (see FIG. 33). A close-up view of the jaws 2621 and 2622 being loaded with occluders from the cartridge is shown in FIGS. 28-30. The applier includes another jaw element 2623 for delivering the transfixing rod 2613 and locking the two elements 2611 and 2612 together, as shown in FIGS. 34-36. The jaws are long enough so that the part of atrial appendage that is to be occluded can fit within the jaws in front of the fulcrum of the two jaws. The length of the applier arms 2621 and 2622 is long enough to easily reach the targeted parts of the heart exposed through a surgical midline stenotomy.

In embodiments, the clip applier 2620 may apply a different delivery or clamping force on each occluder device (i.e., two locking clips with built in rod) or two locking clips and pin, such that one occluder may be locked with very little separation between the proximal and distal occlusion elements, and a second occluder device will be deployed across thicker tissue at the same time with a larger separation between the proximal and distal elements. Sequential deployment of each of the clips occluding different tissue thicknesses is also possible.

The applier 2620 may be spring loaded, to accommodate the different thickness of tissue, or to apply different forces to lock the thicker and thinner tissues together. In another embodiment, the delivery jaws loaded with the occluding devices 2610 can be rotated (swiveled) so that occluders can be placed at any chosen point of the area where the edges of the heart (ventricle or atrium) need to be sealed together.

Delivery of the elements occurs when the jaws of the occluding element are placed at the chosen point, and the two edges to be sealed are compressed with delivery of the two occluding elements. The occlusion elements lock together transfixing the tissue.

Additional Embodiments of Occluder Devices

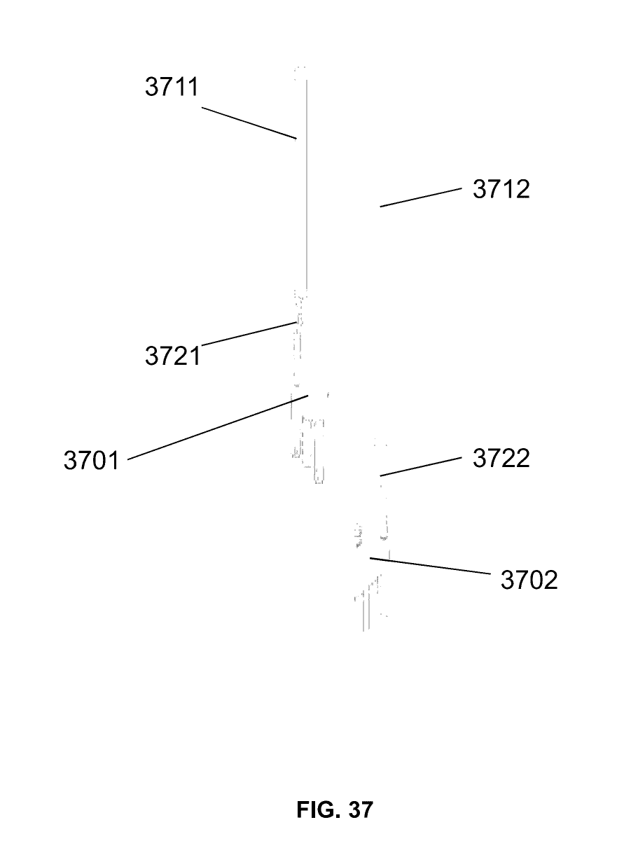

Occluder devices can take on various embodiments, as has been described. Additional embodiments of occlusion devices are shown in FIGS. 37-39.

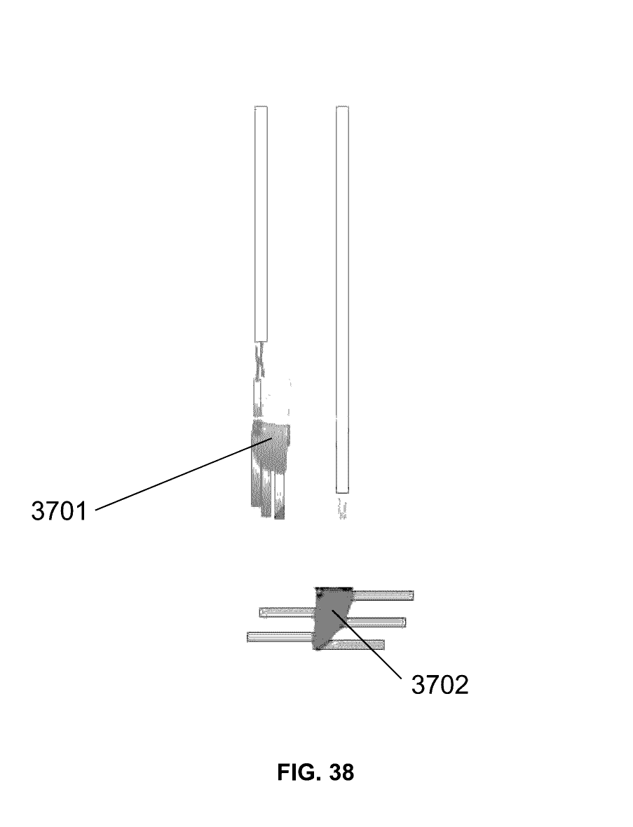

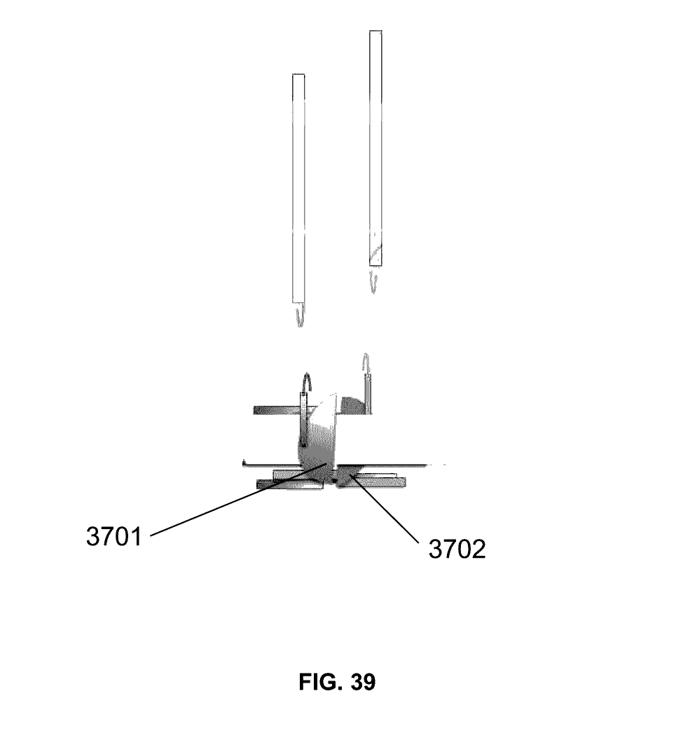

In the embodiment shown in FIGS. 37-39, the delivery tube has sufficient diameter or size to allow deployment of two solid occluders that can be moved independently from each other within the delivery device. Each of the proximal occluder 3701 and distal occluder 3702 has its own pushrod 3711 and 3712, which may also be a guidewire, which connects to its respective occluder element by a locking and release element 3721 and 3722 which can be activated controllably by the user from the proximal end of the delivery device. As can be seen in FIG. 37, the two occluder elements have room to slide past one another within the delivery tube. FIG. 38 shows the deployment of the distal occluder. FIG. 39 shows deployment of the proximal occluder, which latches together with the distal occluder as shown. The pushrods 3711 and 3712 are released via the release elements 3721 and 3722. The release elements are depicted as hooks in FIG. 39.

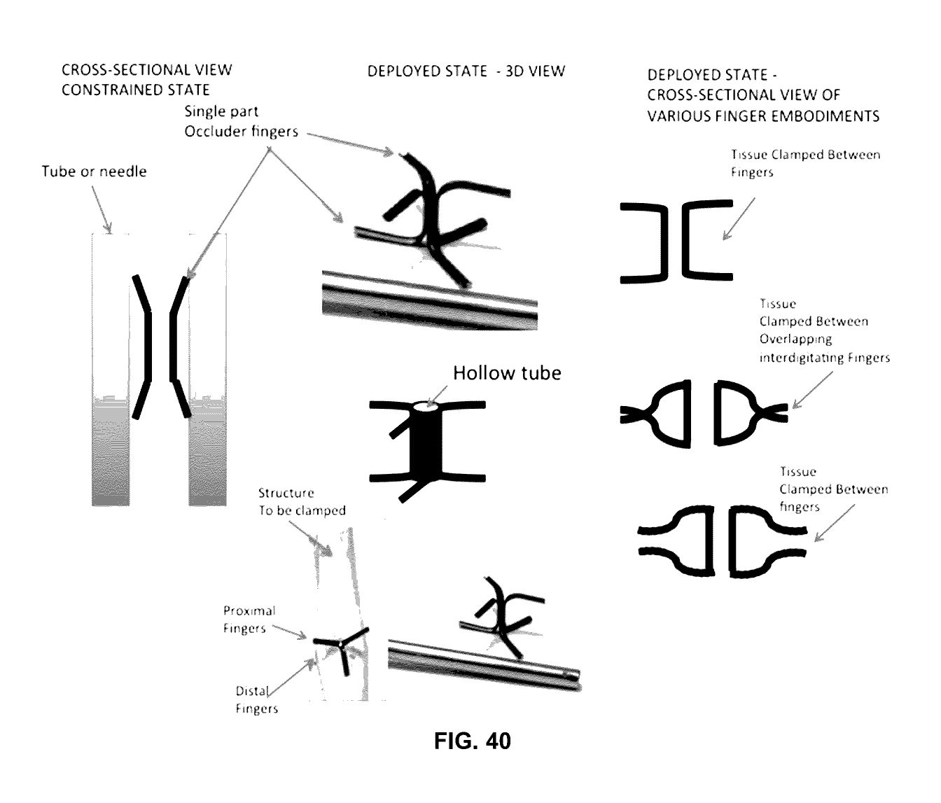

In another embodiment, an occluder can be a single-piece device with a hollow center. It can be constructed, for example, out of a hollow tube with slits cut into each end of the tube to form a plurality of legs. The occluder can be made of nitinol or another superelastic material and can be fixed in its open state (shown in FIG. 40), which corresponds to its configuration when deployed from a delivery tube. In some embodiments, the occluder is made of bioabsorbable materials such as, PGA (Polyglycolic acid), PLLA (Poly-L-Lactide), PGLA (Polyglycolic acid-co-poly-L-lactic acid), Polydioxanone (PDO), PGA (Polyglycolic acid), and PLLA (Poly-L-Lactide).

As shown in FIG. 40, the legs can be configured with differing levels of finger overlap in the deployed or relaxed state. However, each can be constrained into a more linear configuration for loading into the delivery tube or needle. The occluders can vary in the number of fingers ranging from 2 to 5 or more.

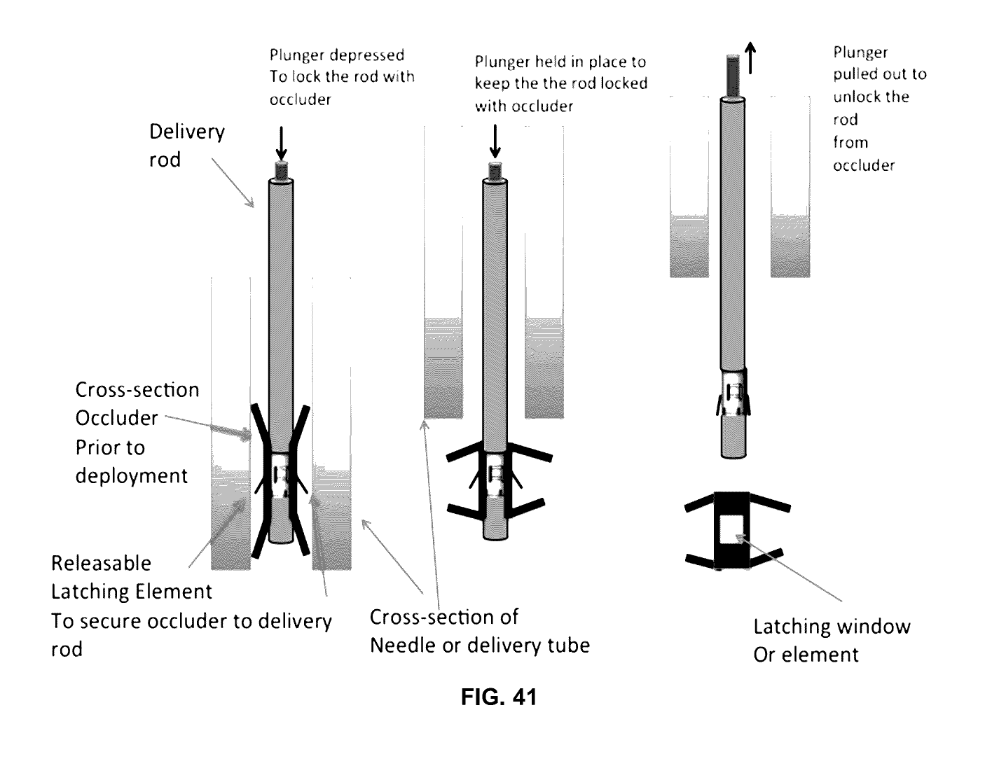

The occluder may be delivered with a rod having an operator-controllable expanding or reversible locking region at the end of the rod, as shown in FIG. 41. The pushrod and locking element constitute a single unit that grips the occluder to be delivered, holding the occluder element throughout deployment and until deployment is completed whereupon the rod is released from the occluder element. The expansion and locking can be induced by pulling the locking rod into a surrounding sheath, thereby expanding the diameter of the sheath. In other embodiments, the end of the rod can be expanded by inflating it by pumping air or saline into a polymeric biocompatible compliant material that forms a balloon-like structure at the end of the delivery rod. In other embodiments, such as that depicted in FIG. 41, a plunger inside the delivery rod activates locking flaps that fit into latching windows in the occluder element, and hold the occluder connected to the delivery rod through the delivery process. Upon delivery of the occluder, the plunger is released, and the flaps collapse so they are no longer deployed in the occluder windows and the rod can be removed. In some embodiments, an electrical signal detector is connected to the delivery rod for detecting whether the occluder is in contact with a nerve. If a nerve is detected, prior to full deployment of occluder, the occluder may be redeployed to a region without a nerve present.

Several hollow occluders can be disposed within a delivery tube, as shown in FIG. 42. An occluder is delivered utilizing the locking rod, which delivers and holds the occluder being deployed. The delivery steps are shown in FIG. 42. The needle pierces the two walls of a tubular structure. The other occluders are constrained in the cylinder, needle or delivery tube by friction with the walls, or by a stopping mechanism, not shown in the figure. The single occluder elements are partially hollow, enabling the delivery rod to pass through them, as shown in cross-sectional views in the figure. The delivery rod is pushed down toward the needle opening to deliver the distal fingers of the occluder. Once the distal fingers are delivered to the distal side of the tubular structure, the needle together with the delivery rod are pulled back so the tubular structure compresses. Then the needle is retracted, while the occluder delivery rod is held fixed, and the proximal occluder fingers are deployed. Next, the delivery rod locking element is unlocked, or reduced in size to unlock the occluder from the delivery rod. The delivery rod is drawn back into a position in the center of the next occluder device, so that it can be deployed by the same process. The process can be repeated for as many occluders as there are in the device.

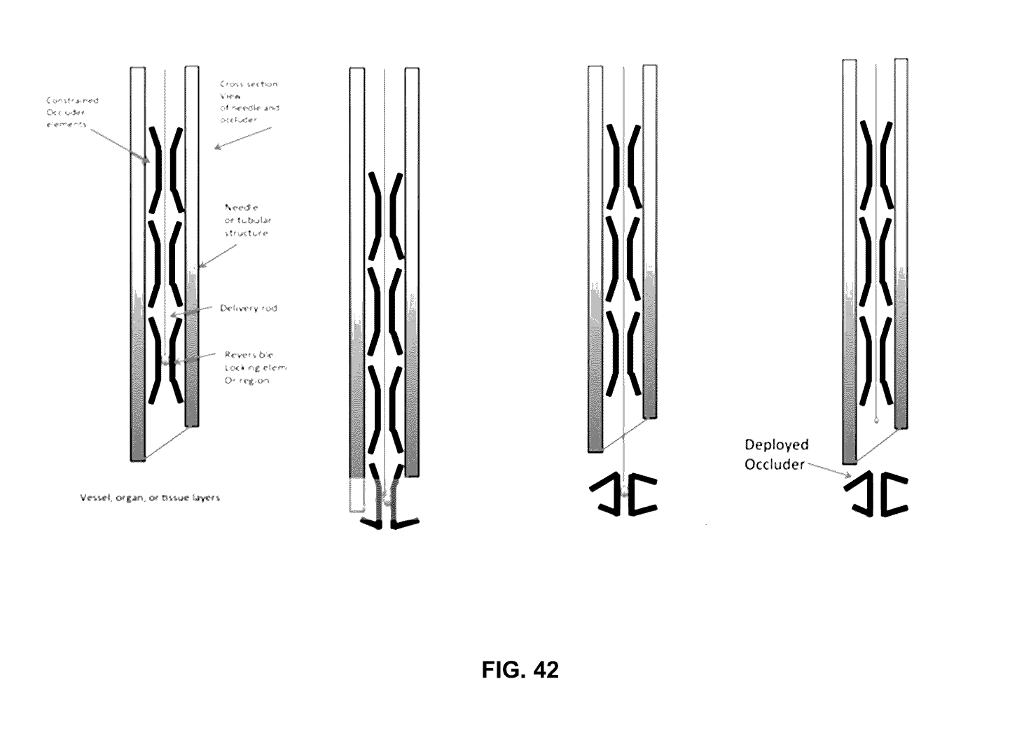

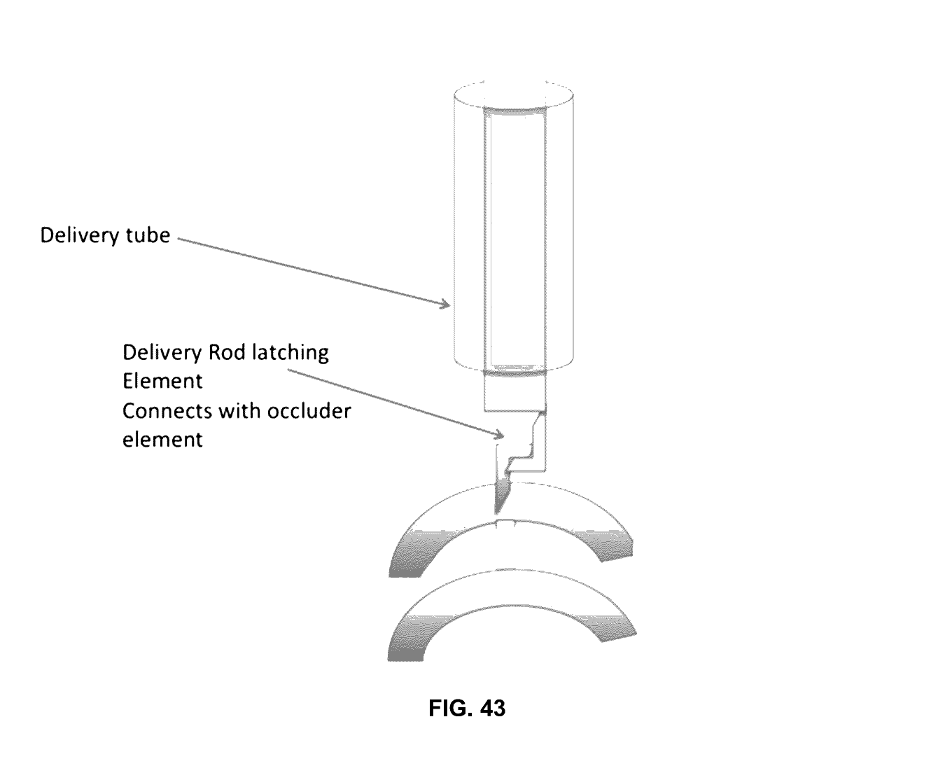

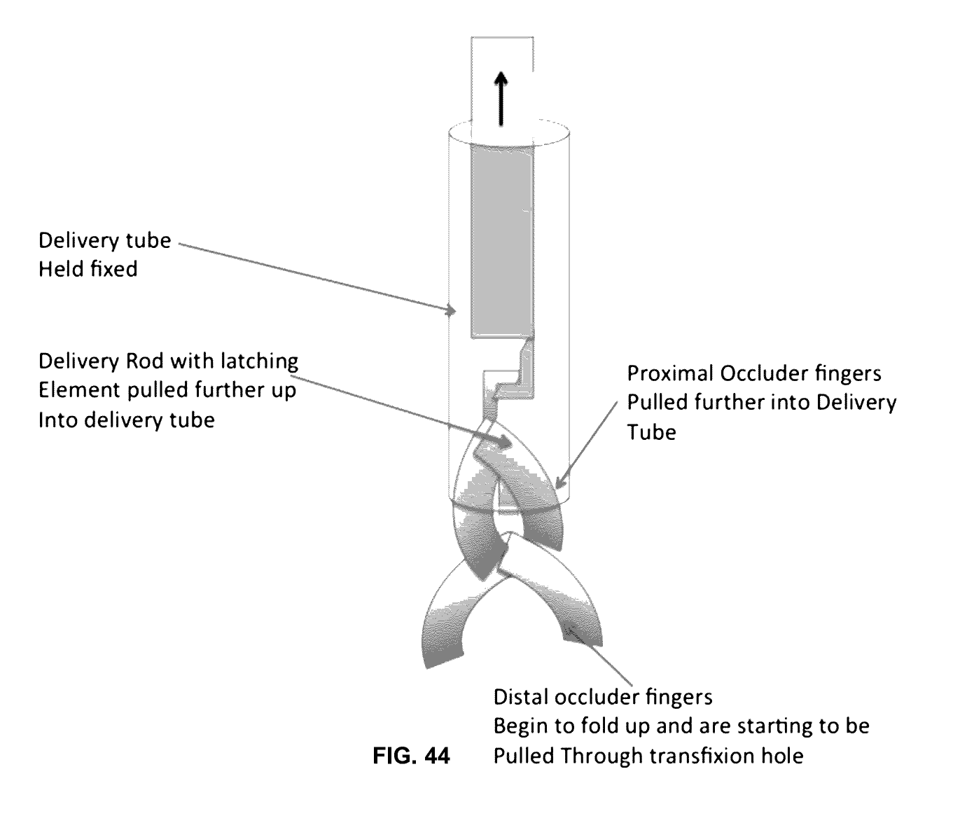

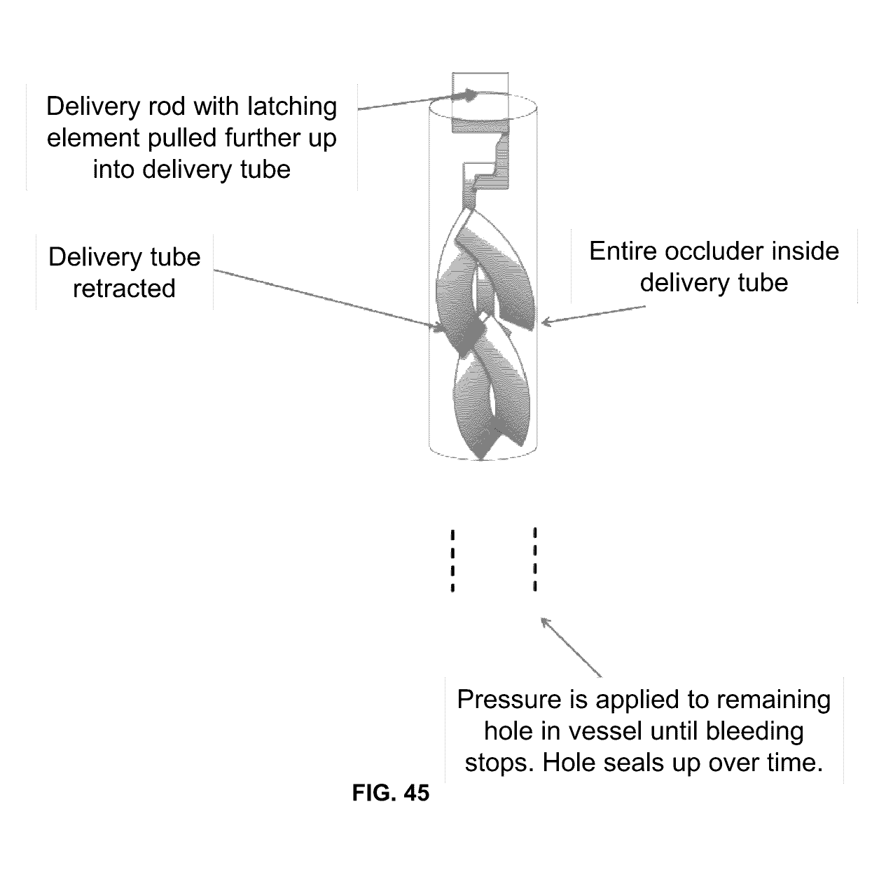

Another embodiment of an occluder is shown in FIGS. 43-45. In this embodiment, the occluder is reversible. In other words, it can be deployed and then later drawn back into a delivery tube and removed. FIG. 43 shows a reversible occluder deployed occluding a vessel. A delivery rod latch element is engaged with a corresponding latching element on the occluder. The delivery tube is placed in contact with the external surface of the occluder. The delivery rod is then pulled back into the delivery tube, and due to the shape of the occluder, the occluder legs bend inward as they are pulled into the delivery tube, as shown in FIG. 44. The occluder is thus extracted while the tubular structure is held primarily static, as shown in FIG. 45. Pressure is applied to the remaining hold in the vessel until bleeding stops. The hole seals itself over time.

In other embodiments envisioned, the occluder fingers may be magnetic and deployed on either side of tubular structure or tissue to be occluded, without the need for a central, transfixing rod. In other embodiments, the magnetic fingers, which will attract each other across the tubular structure or tissue, may include a transfixing rod. In other embodiments the occluder may be composed of at least one circular magnetic disk.

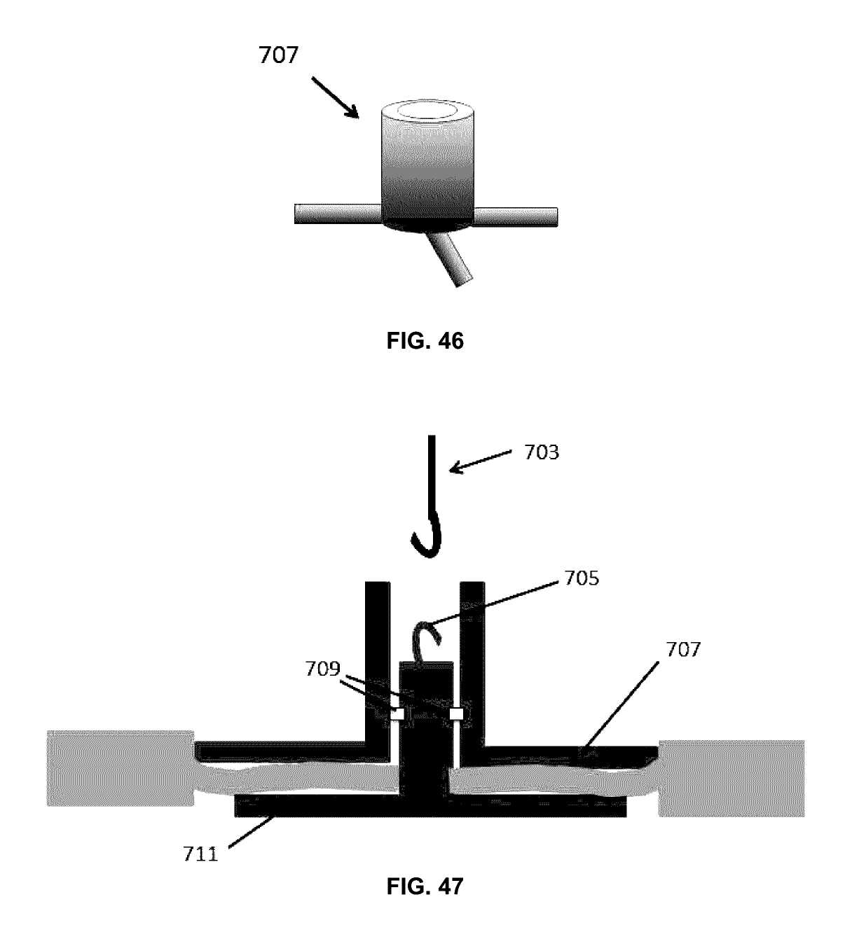

In other embodiments the occluder element has a protective sheath, as shown in FIGS. 46-49.

FIG. 46 shows a proximal occluder element 707. FIG. 47 shows a delivery rod 703, or push rod, or guide wire. For illustration purposes, other surrounding sheaths and delivery components are not shown here but may be present as well. Attachment of the distal occluder locking element 705 or attachment element to the delivery rod or guide wire 703 is mostly protected, or encircled, by the proximal occluder element 707 to minimize any contact the locking or attachment may have with the surrounding tissue and to protect any fragile tissue or organs or vessels from undesired contact. Locking elements 709 may be present to lock the proximal occlusion element 707 and the distal occlusion element 711.

The embodiment of FIGS. 46 and 47 may also be a single part occluder, without the need for two parts to lock together. But again, the attachment mechanism for the delivery device is mostly protected from surrounding tissue by being located within the occluder device.

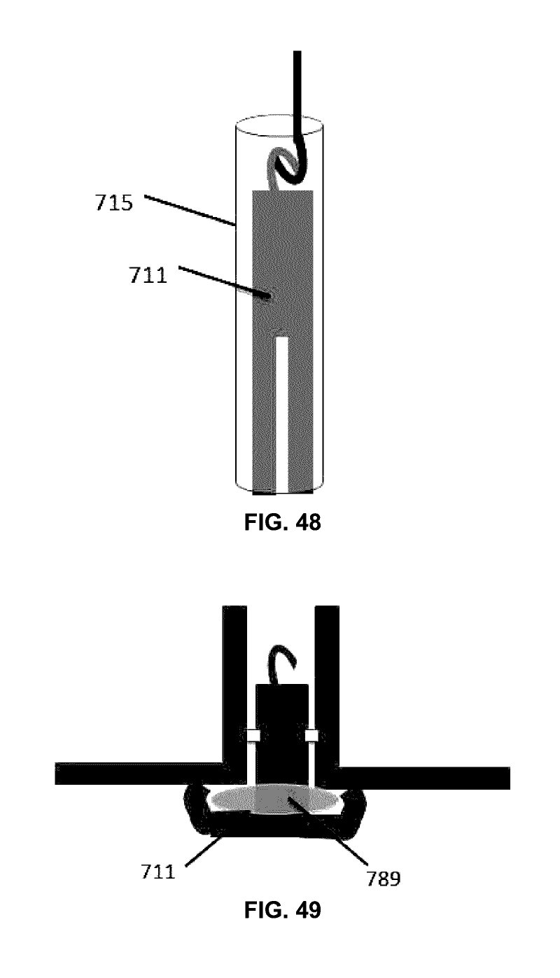

FIG. 48 shows a constrained distal occluder element inside a delivery tube 715, with the delivery rod 703 connected to the distal occluder locking element 705. FIG. 49 shows the released distal occluder element that at least in part wraps around a vessel or structure to be occluded 789.

In some embodiments, both the proximal occlusion element and the distal occlusion element are hollow. When the device is in place, it thereby creates a sealed opening from the outside of a structure to the inside. The device can be used to create an access port or a similar type of opening to allow the entry or exit of a fluid or pressure. The port can also be for facilitating a surgical procedure. The device can therefore be used for a variety of procedures, including rapid ileostomy, loading a feeding tube, a hydrocephaly shunt to drain fluid, tracheostomy, chest wall drainage, arteriovenous fistula shunt, or creating a dialysis port. The device can be used to create an airway access or a venous access.

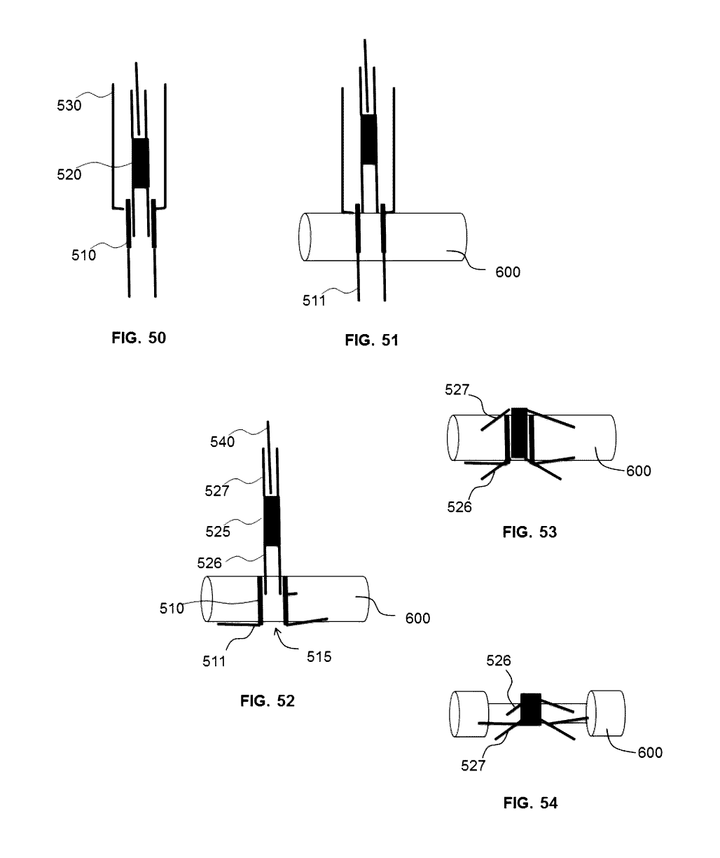

FIGS. 50-54 show another embodiment of an occluder for use with the invention. The occluder comprises a distal implant 510 and a transluminal implant 520. The implants are delivered in much the same way as other two-piece implants that have been described herein. Both implants are loaded into a delivery tube 530 in their compressed configurations. As shown in FIG. 51, the distal implant 510 is delivered through the two walls of a tubular structure 600 and out to the far side of the tubular structure 600. Upon release from the constraints of the delivery tube 530, the expanding region 511 of distal implant 510 expands to its relaxed state. The delivery tube 530 is drawn back through the tubular structure 600.

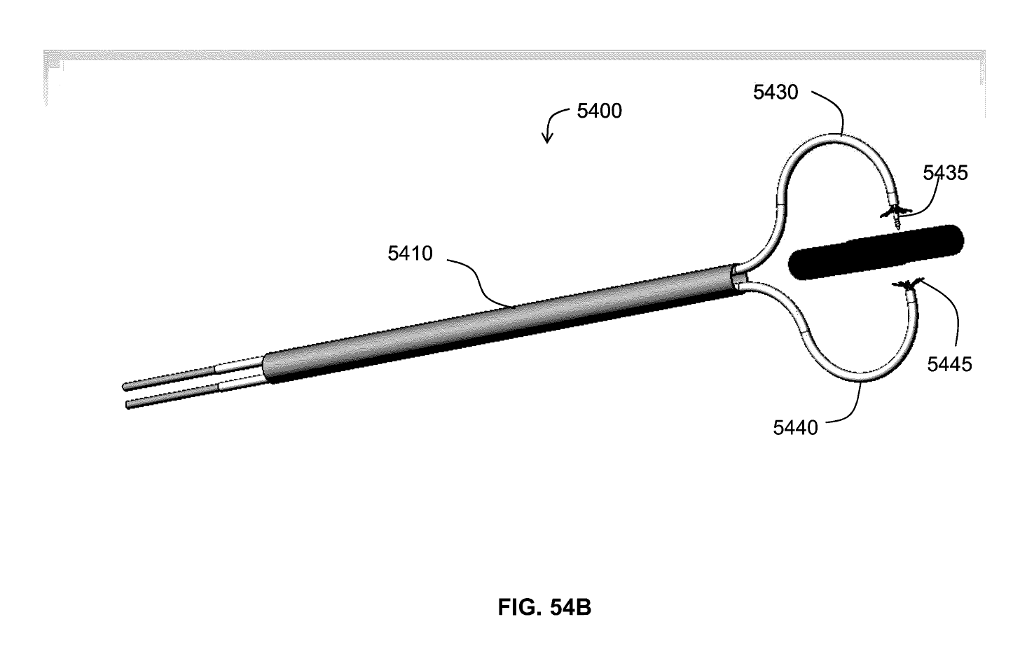

As seen in FIGS. 52-54, the transluminal implant 520 fits within an open space 515 defined by the distal implant 510. The transluminal implant 520 includes an implant body 525, a distal expanding region 526 and a proximal expanding region 527. The transluminal implant 520 is pushed with a pushing member 540 into the space 515 defined by the distal implant 510 until the implant body 525 is substantially aligned with the space 515 defined by the distal implant 510 (FIG. 53). The distal expanding region 526 expands upon being pushed through. The proximal expanding region 527 expands when the delivery tube 530 is further withdrawn, thereby releasing the proximal expanding region 527. As seen in FIGS. 53 and 54, the expanded shape of the transluminal implant 520 holds it in place with respect to the distal implant 510, thereby locking the implants together. Thus unlike some other two-piece occluders described herein, in the two-piece occluder of FIGS. 50-54 the locking occurs outside of the overlap region of the occluders.

FIG. 54B shows another embodiment of an occluder delivery device 5400. The delivery device 5400 has a sheath 5410 and two delivery tubes. The proximal delivery tube 5430 delivers a proximal occluder 5435 and the distal delivery tube 5440 delivers a distal occluder 5445. The delivery tubes 5430 and 5440 are configured to extend outward from the longitudinal axis of the sheath 5410 and bend back inwards so that the distal ends of each delivery tube point towards each other. That configuration allows the delivery tubes to avoid interfering with a proximal vessel when attempting to occlude a distal vessel. The occluder is thus delivered in an orientation perpendicular to the longitudinal axis of the delivery sheath 5410. Delivery device 5400 allows percutaneous delivery when other delicate structures are located near the structure to be occluded. In the embodiment shown, the occluders 5435 and 5445 lock together

Pullback Method of Occluder Delivery

A preferred method for delivering occluders to a tubular structure involves compressing or flattening the tubular structure, such as by pulling back on the distal implant, before fully deploying the occluder device. The methods described herein can be employed laparoscopically, percutaneously, and in open surgical procedures. The method involves penetrating the tubular structure with a delivery device such as a needle and releasing a distal occlusion member on the far side of the tubular structure. When it is released from the delivery device, it assumes an expanded shape that is ideal for applying a force against the far side of the tubular structure.

Once the distal member is deployed in its expanded shape, the delivery device (while still coupled to the distal member) is pulled proximally, causing the distal member to exert a force on the far side of the tubular structure to bring the far side of the tubular structure into contact with the near side of the tubular structure, thereby closing the lumen between the far side and the near side. The tubular structure is thus caused to assume a compressed or flattened shape. The pulling of the far side of the tubular structure therefore occludes the lumen.

A proximal member is delivered to the near side of the collapsed tubular structure. The proximal member is locked together with the distal member, and together they apply a force directed at opposing walls of the tubular structure, thereby holding the tubular structure in the compressed state. The proximal member can thus be delivered in a controlled manner to the tubular structure that is already compressed. The occlusion that results from the pulling of the distal implant allows the two implants to come together more easily.

In other embodiments, the flattening of the tubular structure is achieved through the use of a separate instrument to compress the tubular structure before connecting the distal and proximal implants. The instrument can be some type of engaging member, such as a clamp that compresses the tubular structure, thereby occluding the lumen. The engaging member can be a laparoscopic device. It can be connected to the delivery device or it can be separate.

In certain aspects, the invention provides a method for occluding a tubular structure such as a blood vessel. The method includes penetrating a tubular structure with a hollow delivery device and deploying a distal implant on a far side of the tubular structure. The method further involves pulling back with the hollow delivery device to cause the distal implant to compress the tubular structure, thereby occluding the tubular structure. The method also involves deploying a proximal implant on the near side of the tubular structure to secure the tubular structure in its compressed state.

In certain embodiments, the the hollow delivery device is a needle. In other embodiments, the distal implant is made of a super elastic material that is configured to assume a compressed state for loading into the delivery device and an expanded state when released from the delivery device, and the proximal implant is made of a super elastic material that is configured to assume a compressed state for loading into the delivery device and an expanded state when released from the delivery device.

In embodiments, deploying the distal implant is done by pushing the distal implant out of the delivery device to cause it to assume its expanded state. In certain embodiments, the proximal implant is pushed out of the delivery device by a proximal implant delivery tube, and the distal implant is pushed out of the delivery device by a distal implant delivery tube disposed concentrically within the proximal implant delivery tube.

In other aspects, the invention provides a method for occluding a tubular structure such as a blood vessel. The method includes providing a hollow needle containing an occluder and providing an engaging member. The method further includes manipulating the engaging member against a tubular structure to compress a region of the tubular structure. The method also includes penetrating the compressed region of the tubular structure with the hollow needle. The method also includes deploying the occluder to occlude the tubular structure at the compressed region.

In some embodiments, the occluder includes a distal implant and a proximal implant. The distal implant can be made of a super elastic material that is configured to assume a compressed state for loading into the delivery device and an expanded state when released from the delivery device, and the proximal implant can be made of a super elastic material that is configured to assume a compressed state for loading into the delivery device and an expanded state when released from the delivery device.

In embodiments, deploying the distal implant is done by pushing the distal implant out of the delivery device to cause it to assume its expanded state. The needle may further include a proximal implant delivery tube and a distal implant delivery tube disposed concentrically within the proximal implant delivery tube.

In some embodiments, a vessel is pushed closed by the needle delivery device itself, prior to deployment of the occluder. Depending on the thickness of the needle wall, the outside diameter (OD) of the needle, and the sharpness of the needle, different delivery devices can be designed to cleanly pierce a vessel or to compress the vessel prior to puncturing it. Experiments with needles of various parameters have shown that, thin needle walls (for example, less than 0.15 mm) cause the needle to be more “bouncy” and push the tissue closed before puncturing it. Needles with thickness of around 0.2 mm or greater tend to directly puncture the vessel. Relative ratios of OD to wall thickness can impart different qualities on the delivery needle.

Although the method is described with respect to a tubular structure, such as a vein or a vessel, the method can be applied to other similar uses as well, including closing a hole in an anatomical structure or connecting two structures together.

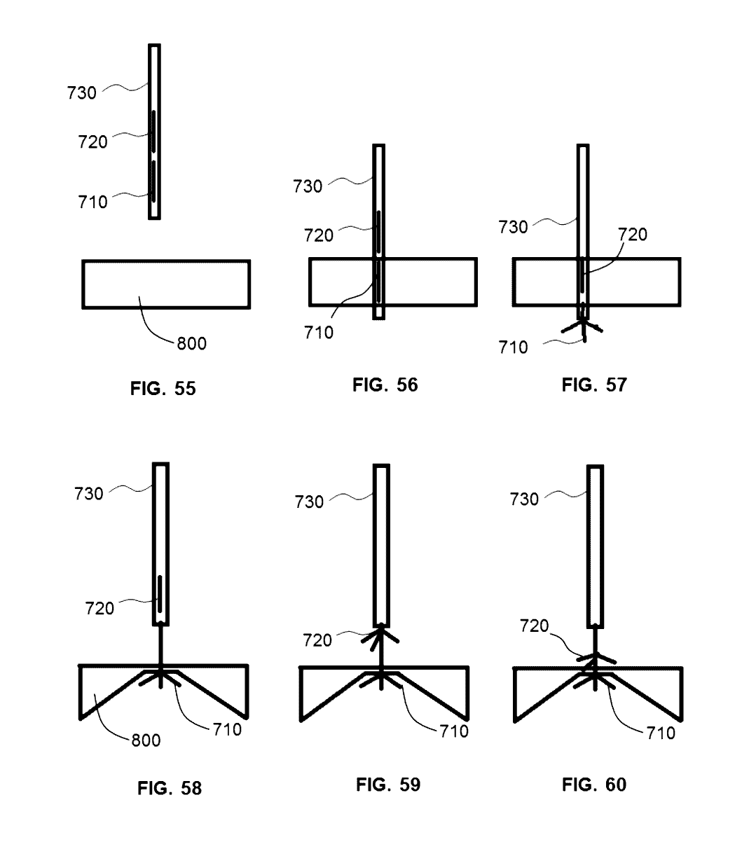

FIGS. 55-60 show a method of occluding a vessel wherein the vessel is pulled proximally by the distal implant in accordance with the present invention. FIG. 55 shows a delivery needle 730 containing a distal implant 710 and a proximal implant 720 in their compressed linear configurations. The delivery needle 730 is approaching a blood vessel 800 to be occluded. In FIG. 56, the delivery needle 730 has penetrated the first and second walls of the vessel 800. In FIG. 57, distal implant 710 has been pushed out of the distal end of the needle 730 by a pushing apparatus (not shown), such as an implant delivery tube or other pushing member described herein. The distal implant 710 assumes its diametrically-expanded shape upon release from the lumen of the needle 730.

In FIG. 58, with the distal implant 710 deployed on the far side of the vessel 800, the delivery needle 730 is pulled proximally by the user. Thus the distal implant 710 imparts a proximal force on the vessel, causing it to compress or flatten at the location to be occluded. While the vessel 800 is in the flattened position, the proximal implant 720 is pushed out of the needle 730, thereby achieving its expanded configuration (FIG. 59). The proximal implant 720 is moved towards the distal implant 710 until the two implants lock together around the compressed blood vessel 800, thereby fixing the blood vessel 800 in the occluded state (FIG. 60). The delivery needle 730 can then be withdrawn, leaving only the two implants in place occluding the vessel.

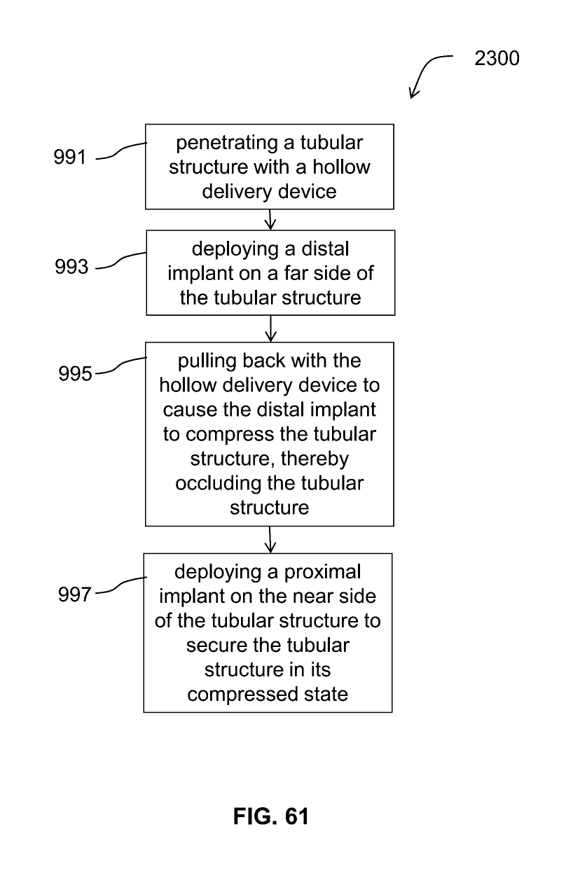

FIG. 61 shows the steps of a method 2300 of occluding a tubular structure according to the present invention. The method includes penetrating 991 a tubular structure with a hollow delivery device and deploying 993 a distal implant on a far side of the tubular structure. The method further involves pulling back 995 with the hollow delivery device to cause the distal implant to compress the tubular structure, thereby occluding the tubular structure. The method also involves deploying 997 a proximal implant on the near side of the tubular structure to secure the tubular structure in its compressed state.

### Example 1: Sequence of Occlusion in an LAA Procedure

In a demonstration of certain methods of the present invention, occlusion of an LAA was achieved by the following steps:
1. The approximate border of the LAA was marked out and measured for length.
2. The LAA was held with two forceps, elevating the LAA to improve the angle of delivery.
3. Counter pressure was applied to the needle entry point with a forceps (retractor) on the surface to the LV provided for simple accurate deliver through both layers.
4. The atrium was opened and the occlusion line examined from within and on inspection was shown to be competent. Of note, no elements, including the connecting struts of the occlusion devices, could be observed within the suture line.

The results of the demonstration of the method were positive. The design of the occlusion elements and the flexibility properties of the nitinol were well demonstrated. No tearing of the LAA walls was noted. The interdigitation of the elements were again noted to be a critical and significant element of the occlusion efficiency of the occluder as well as reducing the occlusion line length and thus reducing the number of occluders required. Clearly enlarging the size of the occluder (enlarging the size of the “fingers” of the occluding elements) would minimize the number of occluders required.

The animal study, as shown in FIGS. 22-34 confirms the feasibility of use of the two part of one part occluder device for LAA occlusion and/or amputation.

### Example 2: Treatment of Mitral Insufficiency

Another use for the disclosed devices is to treat mitral insufficiency (or mitral regurgitation or mitral incompetence), which is a disorder of the heart in which the mitral valve does not close properly when the heart pumps out blood. Occluders of the present disclosure can be delivered to clamp together the leaflets of the mitral valve, thereby stabilizing the mitral valve and preventing the abnormal leaking of blood backwards from the left ventricle, through the mitral valve, into the left atrium, when the left ventricle contracts.

### Example 3: Use of Occluders for Management of Varicose Veins by the CHIVA Approach

Varicose veins, estimated to effect approximately 23% of US adults, are part of a spectrum of chronic venous disease and is generally more common in women than men between the ages of 40-80 years. Untreated, varicose veins may eventually progress to severe chronic venous insufficiency with symptoms and other manifestations including lower extremity venous ulceration. The standard of care in the US for the treatment of varicose veins until the turn of the century was surgery with high ligation, stripping of the saphenous vein and phlebectomy of the presenting varicosities. More recently, alternative treatments for varicose veins including endothermal ablation with laser or radiofrequency have replaced surgery as the standard of care together with chemical treatments, sclerosants and chemical adhesives. These procedures have brought the treatment of varicose veins out of the hospital surgical operating rooms and into the outpatient surgical clinics, reducing hospital costs for patient management and minimizing patient morbidity; however, the underlying pathophysiological approach to the treatment of these patients has not changed. It remains aimed at preventing all reflux in the lower extremity veins with occlusion of a long segment of the saphenous vein from the sapheno-femoral junction, occlusion of large tributaries, and removal or hook-phlebectomy of the superficial varicosities.

In the early 1990's Claude Franceschi, described an alternative approach, saphenous sparing, to treat the venous insufficiency associated with varicose veins. This approach, known as “CHIVA” (Cure conservatrice et Hemodynamique de L'Issufsance veineuse en Ambulatoire or conservative hemodynamic cure for varicose veins), unlike the surgical and endovascular approaches, does not aim at elimination of all reflux in the lower extremity veins, but directs the flow of the “refluxing” blood into the deep veins of the lower extremity. CHIVA concept ultimately attempts to relieve the pressure in the superficial veins, eliminates the problem of superficial venous hypertension, and the clinical consequences of varicose veins.

This method of treatment has two main requirements, an accurate ultrasonic assessment of the flow patterns in the extremity veins to choose the optimal flow diversions that need to be performed, and accurate open surgical ligation. These minimally invasive surgical procedures require some surgical expertise. In addition, because multiple occlusions are required, they are also time consuming and can be disfiguring. Because of the combination of demanding ultrasonic expertise, and the associated surgical skills required to execute the plan, CHIVA has not been adopted by the majority of venous interventionalists. However, as a result of the expense of the endothermal or chemical ablation devices, there may be a developing interest in the CHIVA technique if simplified.

The occluders and methods described in the present disclosure provide an alternative simple, percutaneous, mechanical method of vessel occlusion with a device that eliminates the need for these time consuming and skilled surgical procedures. This technique will simplify and expedite the CHIVA procedure for the doctor and minimize patient's discomfort and recovery. The occluders can be delivered percutaneously or during open surgical procedures, in vessels up to 12 mm in diameter.

As described herein, occluders of the present invention can be preloaded into an 18G needle of a delivery device, such as the delivery devices described throughout the present disclosure. When deployed, the occluders transfix and occlude the target vessel. The occluder may include two stellate compression elements and a titanium fine strut which connects and locks the compression elements together. The proximal element, which compresses the near wall of the vessel, and distal element, which compresses the far wall of the vessel are made of nitinol, which once deployed, assumes its designated configuration closing off the vessel. The individual legs of the proximal occlusion component may be configured to alternate with and interdigitate with the individual legs of the distal occlusion component. This interdigitation is important as it obviates the problem with different vessel wall thicknesses and creates a zone of occlusion that surrounds the site of penetration, preventing leaks at the needle entry site.

In embodiments, the occluder deliver device delivers an occluder under ultrasound guidance. Clear identification of the vessel is essential in order to transfix the vessel by passing the needle through both walls of the vessel. A small opening in the transfixing needle allows the escape of blood as the needle enters into the lumen of the vessel to confirm accuracy of needle placement within the targeted vessel, as well as confirm the identity of the vessel, artery versus vein. Once the vessel is transfixed, further ultrasound guidance is not required. The two occluding elements are released, locked together and the vessel occluded. The delivery device is then detached and withdrawn.

In animal studies using a porcine model, 30 ultrasound guided percutaneous occlusions were successfully performed in vessels ranging from 2-12 mms. These vessels include the proximal femoral vessels, distal (superficial) femoral arteries and veins, carotid arteries and jugular veins. For the larger vessel size, >7 mm, a 2nd occluder clip was necessary to completely occlude the targeted vessel. In certain embodiments, the size of the clip can be determined by need; thus, for a larger vessel, a larger clip can be used.

All targeted vessels in this study were successfully occluded and occlusion confirmed by Duplex ultrasound as well as open surgical exposure. In addition, no injury to any of the adjacent structures was identified.

The occluder with its ability to accurately occlude vessels percutaneously under ultrasound guidance simplifies the technique of the CHIVA procedure. This novel occlusion device eliminates the need for “open” surgical procedures and thus minimizes the invasiveness of the CHIVA procedure. The simple, mechanical occlusion devices disclosed herein will allow a wider spectrum of caregivers to adopt the CHIVA approach, which is a more conservative approach with saphenous vein preservation for the treatment of symptomatic varicose veins and chronic venous insufficiency. It will also improve patient comfort and acceptance of the procedure, speed recovery and reduce procedural morbidity and the associated health care costs.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for occluding an atrial appendage, the method comprising:
deploying an occluder device transfixing a first wall and a second wall of an atrial appendage.

2. The method of claim 1, wherein the occluder device comprises a distal implant and a proximal implant configured to lock together.

3. The method of claim 1, wherein the occluder device is configured to assume a diametrically-reduced configuration for loading into a delivery tube and a diametrically-expanded configuration for deployment adjacent to the atrial appendage.

4. The method of claim 3, wherein the delivery tube is a needle or a laparoscopic device.

5. The method of claim 1, further comprising encircling the atrial appendage with a sling.

6. The method of claim 5, further comprising occluding the atrial appendage with the sling before deploying the occluder.

7. The method of claim 1, further comprising deploying a plurality of occluder devices.

8. The method of claim 7, further comprising amputating the atrial appendage after deployment of the plurality of occluders.

9. The method of claim 5, wherein the sling comprises a synthetic polymer or animal tissue.

10. The method of claim 5, further comprising using the sling to lift the atrial appendage away from other heart tissue.

11. The method of claim 10, further comprising placing a protective device between the atrial appendage and the other heart tissue.

12. The method of claim 1, further comprising deploying a plurality of occluder devices transfixing the first wall and the second wall.

13. The method of claim 12, wherein the plurality of occluders are deployed adjacent to an ostium of the atrial appendage.

14. The method of claim 13, wherein the plurality of occluder devices are deployed in a non-linear orientation.

15. The method of claim 1, wherein the occluder device comprises a central post with a plurality of flexible legs extending radially from a proximal end of the post and a plurality of flexible legs extending radially from a distal end of the post.

16. The method of claim 15, wherein each set of legs forms a stellate configuration.

17. The method of claim 15, wherein the legs are configured to compress a tissue disposed therebetween.

18. The method of claim 17, wherein the proximal legs and the distal legs interdigitate with each other.

19. The method of claim 18, wherein the proximal legs and the distal legs together form a region of compression that completely surrounds the central post.
